SEQUENCE LISTING (2 sequences)

SEQ ID NO: 1 - DNA, 6096 bp, Homo sapiens
atggtagaccctgtgggtttcgcggaggcctggaaagcgcaatttcctgactccgaacctccccgaatggaacttcggtctgtaggagacatcgagcaggaactagaacgttgcaaggcgtcaattcgcagactggagcaggaggttaaccaggagaggtttcgtatgatatacctgcaaacattgctagcgaaggagaagaaatcctacgatcgccaacggtggggatttcgacgtgctgctcaggcccctgacggcgcaagtgaaccccgggcatccgcatcaaggccccaacctgctccggccgatggcgccgacccgccccccgctgaggaacctgaagctcgccccgacggcgaaggctcacccggaaaggcacgaccaggcaccgcaagaagaccaggggctgcagctagcggagaaagagacgaccgaggtccaccagcctctgtcgccgccctgcgctccaattttgagaggatcaggaaaggacatgggcagccaggcgccgatgccgaaaagccattctatgtgaatgtggagttccaccacgagaggggcctggtcaaggttaacgataaagaggtgagcgatcgcatcagttctcttggctctcaggccatgcagatggagagaaaaaaaagccagcatggagccggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagagtgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctactcgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 2 - DNA, 6096 bp, Homo sapiens
atggtagaccctgtgggtttcgcggaggcctggaaagcgcaatttcctgactccgaacctccccgaatggaacttcggtctgtaggagacatcgagcaggaactagaacgttgcaaggcgtcaattcgcagactggagcaggaggttaaccaggagaggtttcgtatgatatacctgcaaacattgctagcgaaggagaagaaatcctacgatcgccaacggtggggatttcgacgtgctgctcaggcccctgacggcgcaagtgaaccccgggcatccgcatcaaggccccaacctgctccggccgatggcgccgacccgccccccgctgaggaacctgaagctcgccccgacggcgaaggctcacccggaaaggcacgaccaggcaccgcaagaagaccaggggctgcagctagcggagaaagagacgaccgaggtccaccagcctctgtcgccgccctgcgctccaattttgagaggatcaggaaaggacatgggcagccaggcgccgatgccgaaaagccattctatgtgaatgtggagttccaccacgagaggggcctggtcaaggttaacgataaagaggtgagcgatcgcatcagttctcttggctctcaggccatgcagatggagagaaaaaaaagccagcatggagccggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtgagctggacttggaaaagggcttggagatgagaaaatgggtcctgtcgggaatcctggctagcgaggagacttacctgagccacctggaggcactgctgctgcccatgaagcctttgaaagccgctgccaccacctctcagccggtgctgacgagtcagcagatcgagaccatcttcttcaaagtgcctgagctctacgagatccacaaggagttctatgatgggctcttcccccgcgtgcagcagtggagccaccagcagcgggtgggcgacctcttccagaagctggccagccagctgggtgtgtaccgggccttcgtggacaactacggagttgccatggaaatggctgagaagtgctgtcaggccaatgctcagtttgcagaaatctccgagaacctgagagccagaagcaacaaagatgccaaggatccaacgaccaagaactctctggaaactctgctctacaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagcaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagagtgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaataaggaagatgatgagtctccggggctctatgggtttctgaatgtcatcgtccactcagccactggatttaagcagagttcaaaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacacgagaaccaccattctatatcatcattgagttcatgacttatggaaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctactcgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

(19) United States
(12) Patent Application Publication
(10) Pub. No.: US 2020/0037588 A1
(43) Pub. Date: Feb. 6, 2020

(54) METHODS OF A DROSOPHILA MODEL FOR CHRONIC MYELOID LEUKEMIA (CML) TREATMENT

(71) Applicant: American University of Beirut, Beirut (LB)

(72) Inventors: Rihab Nasr, Beirut (LB); Margret Shirinian, Beirut (LB); Amani Al-Outa, Aramoun (LB); Ali Bazarbachi, Beirut (LB); Marwan El-Sabban, Beirut (LB)

(21) Appl. No.: 16/529,062

(22) Filed: Aug. 1, 2019

Related U.S. Application Data
(60) Provisional application No. 62/713,096, filed on Aug. 1, 2018.

(51) Int. Cl. A01K 67/033

(52) U.S. Cl. CPC A01K 67/0339; A01K 2227/706; A01K 2207/15; A01K 2267/0393; A01K 2267/0331; A01K 2267/0381; A01K 2207/05

(57) ABSTRACT

As disclosed herein, the invention relates to a method of screening for a therapeutic for chronic myeloid leukemia. In an aspect, the invention relates to transgenic Drosophila. In an aspect, the invention relates to a Drosophila system for screening compounds treating chronic myeloid leukemia.

### CROSS-REFERENCE TO RELATED APPLICATIONS

The present application claims priority to U.S. provisional application Ser. No. 62/713,096, filed Aug. 1, 2018, herein incorporated by references in its entirety.

### SEQUENCE LISTING

The present application contains a Sequence Listing which is submitted electronically in ASCII format and is incorporated by reference in its entirety. Said ASCII copy, created on Aug. 12, 2019, is named 6161-035-1_ST25-2.txt and is 16 KB in size.

### BACKGROUND

The invention generally relates to Drosophila Models.

Chronic myeloid leukemia (CML) is a myeloproliferative neoplasm secondary to a precise cytogenetic abnormality involving a balanced chromosomal translocation between the Abelson murine leukemia (ABL1) gene on chromosome 9 and the breakpoint cluster region (BCR) on chromosome 22. This creates the (BCR-ABL1) fusion gene on chromosome 22 which encodes for a constitutively active tyrosine kinase BCR-ABL1¹. Based on the breakpoints in BCR this translocation results in the formation of (p190, p210 and p230) fusion genes². Overall, 95% of CML patients harbor the p210-kDa fusion protein, BCR-ABL1^{p210 3, 4}. BCR-ABL1 fusion oncoprotein increases the replication machinery and enhances cell growth which is mediated by downstream signaling pathways such as RAS, RAF, JUN Kinase, MYC and STAT⁵⁻¹¹.

CML treatment was revolutionized with the development of tyrosine kinase inhibitors (TKIs) which competitively inhibit the Adenosine triphosphate (ATP) binding site in the BCR-ABL1 kinase domain¹² and hence block the phosphorylation of proteins in the downstream signaling cascade. The first generation TKI (imatinib) showed major therapeutic improvement in the IRIS study (International Randomized Study of Interferon and STI571)¹³. However, imatinib success was outshined by the emergence of resistance caused by point mutations in the ABL1 kinase domain which necessitated the development of second-generation TKIs^{14, 15.} Dasatinib¹⁶⁻¹⁸ and nilotinib¹⁹⁻²¹ revealed faster and deeper molecular responses compared to imatinib in patients with newly diagnosed chronic phase CML. In vitro, dasatinib is more potent than imatinib²²⁻²⁴ and inhibits a wider spectrum of kinases including Src family²⁵. Nilotinib has a greater affinity than imatinib to the ATP binding site in BCR-ABL1 and its spectrum of kinases inhibition involves platelet-derived growth factor receptor (PDGFR) and c-Kit receptors²⁶. Although nilotinib and dasatinib tackled the majority of imatinib-resistant mutations, neither of them targeted the T315I mutation (threonine to isoleucine substitution at position 315 in ABL1 kinase domain) (BCR-ABL1^{T315I})²³. Ponatinib, a third generation TKI, remains the only clinically available drug that is designed to overcome the T315I gatekeeper mutation^{27, 28}. However, post-marketing safety issues with ponatinib involved serious cardiovascular events which led to its temporary suspension and then reintroduction with special patient recommendations^{29, 30}.

In addition to the burden of resistance, therapy with TKIs is hindered by their inability to eradicate leukemic stem cells and hence relapse often accompanies discontinuation of therapy³¹. This fact imparts lifelong therapy with TKIs despite accompanying side effects which results in ever-expanding costs for remission sustainment. Therefore, it seems evident that despite the breakthrough with TKIs, CML remains a pathology that requires vigilant assessment of curative therapeutic interventions.

One simple, multicellular and genetically tractable animal model that is exploited in recent years for modelling human diseases, including cancer, is Drosophila melanogaster³². The present invention attempts to solve this problem as well as others.

### SUMMARY OF THE INVENTION

Provided herein are systems, methods and compositions comprising A Drosophila Model of Chronic Myeloid Leukemia (CML) Treatment. In one embodiment, a chronic myeloid leukemia tailored BCR-ABL1^{p210} and BCR-ABL1^{p210/T315I} fly system comprises testing new compounds with improved therapeutic indices.

The methods, systems, and compositions are set forth in part in the description which follows, and in part will be obvious from the description, or can be learned by practice of the methods, apparatuses, and systems. The advantages of the methods, systems, and compositions will be realized and attained by means of the elements and combinations particularly pointed out in the appended claims. It is to be understood that both the foregoing general description and the following detailed description are exemplary and explanatory only and are not restrictive of the methods, systems, and compositions, as claimed.

### BRIEF DESCRIPTION OF THE DRAWINGS

In the accompanying figures, like elements are identified by like reference numeral among the several preferred embodiments of the present invention.

FIGS. 1A-1A′ are light microscopy images of healthy adult flies from control cross of w¹¹¹⁸ flies to engrailed-GAL4 flies which were cultured on food containing 0.03% DMSO; (FIGS. 1B-1B′) are light microscopy images showing early pupal death of flies expressing BCR-ABL (wild type p210) which were cultured on food containing 0.03% DMSO; (FIGS. 1C-1C′) are light microscopy images showing the reversal of early pupal lethality and enclosure of adult flies expressing BCR-ABL (wild type p210) which were cultured on food containing 20 uM dasatinib. Genotypes indicated are under the control of the imaginal disc promoter engrailed-GAL4. Flies were kept at 25° C.

FIGS. 2A-2A′ are light microscopy images of healthy adult flies from control cross of w¹¹¹⁸ flies to engrailed-GAL4 flies which were cultured on food containing 0.3% DMSO (FIGS. 2B-2B′) are light microscopy images showing early pupal death of flies expressing BCR-ABL (wild type p210) which were cultured on food containing 0.3% DMSO (FIGS. 2C-2C′) are light microscopy images showing reversal of early pupal lethality and eclosure of adult flies expressing BCR-ABL (wild type p210) which were cultured on food containing 100 uM ponatinib. Genotypes indicated are under the control of the imaginal disc promoter engrailedGAL4. Flies were kept at 25° C.

FIGS. 3A-3B are light microscopy images showing the reversal of larval lethality phenotype and first evidence of developing pupae of progeny expressing BCR-ABL (p210 on 100 uM ponatinib. Genotypes indicated are under the control of the imaginal disc promoter engrailed-GAL4. Flies were kept at 25° C.

FIG. 4A is scanning electron microscopy images of adult eyes of flies cultured on 0.03% DMSO or 20 uM dasatinib. The genotypes indicated are under the control of the eye specific promoter GMR-GAL4 eyes. Flies were kept at 18° C. The eye groove, which is a characteristic area at the lower end of the eye characterized by loss of ommatidial facets, is encircled 100. FIG. 4B is a quantification of eye groove area (um²) of progeny cultured on 0.03% DMSO or dasatinib (20 uM) containing food. The bar graph shows the mean of eye groove area (um²)±SD. ****, P<0.0001.

FIG. 5A is a scanning electron microscopy image of adult eyes of flies cultured on 0.3% DMSO or 270 uM ponatinib. The genotypes indicated are under the control of the eye specific promoter GMR-GAL4 eyes. Flies were kept at 18° C. The eye groove is encircled 100. FIG. 5B is a quantification of eye groove area (um²) of progeny expressing BCR-ABL (wild-typep210) cultured on 0.3% DMSO or ponatinib (270 uM) containing food. ****, P<0.0001. The bar graph shows the mean of eye groove area (um²)±SD.

FIG. 6 is a schematic flow chart of one embodiment of the invention.

FIG. 7 is the SEQ ID NO: 1 for the BCR-ABL/P120 DNA sequence.

FIG. 8 is the SEQ ID NO: 2 for the P210 ABL T315I DNA sequence.

FIG. 9 is a schematic diagram of the Phi C31 integrase system.

FIG. 10 is a schematic image of Drosophila PUAStB expression vector: the Drosophila pUAST-attB Drosophila expression vector was obtained from FlyC31: Link is provided here: http://flyc31.org/sequences_and_vectors.php. The sequence of the pUASTattB vector can be found in the GenBank data base under the accession number EF362409.

FIGS. 11A, B, B′, C, D, D′, E, F, F′, G, H, H′, I, J, J′, K, L, L′ show the rough eye phenotype induced by overexpression of human BCRABL1^{p210} and are light (FIGS. 11A, C, E, G, I, K) and scanning electron (FIGS. 11B-B′, D-D′, F-F′, H-H′, L-L′) micrographs of adult Drosophila compound eyes expressing BCR-ABL1^{p210} under the control of the eye specific promoter GMR-GAL4. Flies were raised on 18° C. (11A-B-B′, C-D-D′), 25° C. (FIGS. 11E-F-F′, G-H-H′) or 29° C. (FIGS. 11I-J-J′, K-L-L′). FIGS. 11B′, D′, F′, H′, J′ and L′ are high magnification of the centermost region in FIGS. 11B, D, F, H, J and L respectively (1,370×). GMR-GAL4>w1118 were used as control. Ommatidial facets are depicted in (FIG. 11B′) by (*), misplaced mechanosensory bristles in (FIG. 11D′) depicted by arrowheads and ommatidial fusions in (FIG. 11J′) are shown by arrow. Posterior is to the left. FIG. 11M is a graph representing the quantification of severity of roughness of the adult fly eye using a grading scale. Genotypes indicated are under the control of eye specific promoter GMR-GAL4. Data represents mean±SEM. ****, P<0.0001.

FIGS. 12A, B, B′, C, D, D′, E, F, F′, G, H, H′, I, J, J′, K, L, L′ show the rough eye phenotype induced by overexpression of human BCRABL1^{p210/T315I} and are light (FIGS. 12A, C, E, G, I, K) and scanning electron (FIGS. 12B-B′, D-D′, F-F′, H-H′, JJ′, L-L′) micrographs of adult Drosophila melanogaster compound eyes expressing BCR-ABL1^{P210/T315I} under the control of the eye specific promoter GMR-GAL4. Flies were raised on 18° C. (FIGS. 12A-B-B′, C-D-D′), 25° C. (FIGS. 12E-F-F′, G-H-H′) or 29° C. (FIGS. 12I-J-J′, K-L-L′). FIGS. 12B′, D′, F′, H′, J′ and L′ are high magnification of the centermost region in FIGS. 12B, D, F, H, J and L respectively (1,370×). GMR-GAL4>w1118 were used as control. Ommatidial facets are depicted in (FIG. 12B′) by (*), misplaced mechanosensory bristles in (FIG. 12D′) depicted by arrowheads and ommatidial fusions in (FIG. 12J′) are shown by arrow. Posterior is to the left. FIG. 12M is a graph representing the quantification of severity of roughness of the adult fly eye using a grading scale. Genotypes indicated are under the control of eye specific promoter GMR-GAL4. Data represents mean±SEM. ****, P<0.0001.

FIG. 13 shows the expression of BCR-ABL1^{p210} and BCR-ABL1^{p210/T315I} in the compound eyes and is representative Western blot of the expression of BCR-ABL1 and phosphorylated levels in transgenic adult fly heads expressing BCR-ABL1^{p210} and BCR-ABL1^{p210/T315I} at different temperatures (18° C., 25° C., and 29° C.). Genotypes indicated are under the control of eye specific promoter GMR-GAL4. GMR-GAL4>w1118 were used as control.

FIGS. 14A, A′, B, B′, C, C′, D, D′, E, E′, F, F′, G, G′, H, H′, I, I′, J, J′, K, K′, L, L′ show how imatinib shows a tendency to decrease BCR-ABL1^{p210} mediated eye defect and are scanning electron micrographs (FIGS. 14A-A′, L-L′) of adult Drosophila compound eyes from flies fed on 0.3% DMSO only (FIGS. 14A-A′, D-D′, G-G′, J-J′) or imatinib (FIGS. 14B-B′, C-C′, E-E′, F-F′, H-H′, I-I′, K-K′, L-L′). Posterior is to the left. GMR-GAL4>w1118 were used as control. FIGS. 14A′-L′ are high magnification of the posterior end of the eye in FIGS. 14A-L respectively (692×). Normal development in control flies fed on DMSO (FIGS. 14A-A′, G-G′) or imatinib (FIGS. 14B-B′, C-C′, H-H′, I-I′) is observed. BCR-ABL1^{p210} (FIGS. 14D-D′) and BCR-ABL1^{p210/T315I} (FIGS. 14J-J′) expressing flies fed on DMSO show characteristic defective area with loss of ommatidial facets. Area is marked with a representative dashed line. Feeding low or high dose imatinib to BCR-ABL1^{p210} (FIGS. 14E-E′, F-F′) and BCR-ABL1^{p210/T315I} (FIGS. 14K-K′, L-L′) retained the defective area in the posterior end of the eye marked with a dashed line. Compare to FIGS. 14D-D′ and J-J′ respectively. FIG. 14M is a graph representing the measurement of the posterior eye defect area (μm²). Data represents mean±SEM. ****, P<0.0001.

FIGS. 15A, A′, B, B′, C, C′, D, D′, E, E′, F, F′, G, G′, H, H′ show how dasatinib rescues BCR-ABL1^{p210} driven eye defect and shows target specificity in vivo and are scanning electron micrographs of adult Drosophila compound eyes from flies fed on 0.03% DMSO only (FIGS. 15A-A′, C-C′, E-E′, G-G′) or dasatinib (FIGS. 15B-B′, D-D′, FF′, H-H′). Posterior is to the left. GMR-GAL4>w1118 were used as control. FIGS. 15A′-H′ are high magnification of the posterior end of the eye of FIGS. A-H respectively (692×). Normal development in control flies fed on DMSO (FIGS. 15A-A′, E-E′) or dasatinib (FIGS. 15B-B′, F-F′) is observed. BCR-ABL1^{p210} (FIGS. 15C-C′) and BCR-ABL1^{p210/T315I} (FIGS. 15G-G′) expressing flies fed on DMSO show characteristic defective area with loss of ommatidial facets. Area is marked with a representative dashed line. Ommatidial development in this area was restored with BCR-ABL1^{p210} flies fed on 20 dasatinib (FIGS. 15D-D′). Compare to (FIGS. 15C-C′). BCR-ABL^{p210/T315I} flies showed no restoration of ommatidial development (FIGS. 15H-H′). Compare to (FIGS. 15G-G′). FIG. 15I is a graph representing measurement of the posterior eye defect area (μm²). Data represents mean±SEM. ****, P<0.0001. FIG. 15J is a representative Western blot of the expression of BCR-ABL1 and phosphorylated levels in transgenic untreated and treated adult fly heads. Genotypes indicated are under the control of eye specific promoter GMR-GAL4.

FIGS. 16A, A′, B, B′, C, C′, D, D′, E, E′, F, F′, G, G′, H, H′ show how ponatinib rescues BCR-ABL1^{p210} driven eye defect and are scanning electron micrographs of adult Drosophila compound eyes from flies fed on 0.3% DMSO only (FIGS. 16A-A′,C-C′, E-E′, G-G′) or ponatinib (FIGS. 16B-B′, D-D′, F-F′, H-H′). Posterior is to the left. GMRGAL4>w1118 were used as control. FIGS. 16A′-H′ are high magnification of the posterior end of the eye of FIGS. 16A-H respectively (692×). Normal development in control flies fed on DMSO (FIGS. 16A-A′, E-E′) or ponatinib (FIGS. 16B-B′, F-F′) is observed. BCR-ABL1^{p210} (FIGS. 16C-C′) and BCRABL1^{p210/T315I} (FIGS. 16G-G′) expressing flies fed on DMSO show characteristic defective area with loss of ommatidial facets. Area is marked with a representative dashed line. Ommatidial development in this area was restored with BCR-ABL1^{p210} flies fed on ponatinib (FIGS. 16D-D′). Compare to (FIGS. 16C-C′). BCR-ABL^{p210/T315I} flies showed no restoration of ommatidial development (FIGS. 16H, H′). Compare to (FIGS. 16G, G′). FIG. 16I is a graph representing the measurement of the posterior eye defect area (μm²). Data represents mean±SEM. ****, P<0.000. FIG. 16J is a representative Western blot of the expression of BCR-ABL1 and phosphorylated levels in transgenic untreated and treated adult fly heads. Genotypes indicated are under the control of eye specific promoter GMR-GAL4.

FIGS. 17 A, A′, B, B′, C, C′, D, D′, E, E′, F, F′, G, G′ show how dasatinib and ponatinib rescue BCR-ABL1^{p210} driven eye defect in a dose dependent manner and are scanning electron micrographs of adult Drosophila compound eyes from flies expressing BCR-ABL1^{p210} and fed on 0.03% DMSO (FIGS. 17A-A′), 1 μM (FIGS. 17B-B′), 10 μM (FIGS. 17C-C′) or 20 μM (FIGS. 17D-D′) dasatinib and flies fed on 0.3% DMSO (E-E′), 28 μm ponatinib (FIGS. 17F-F′) or 280 μM ponatinib (FIGS. 17G-G′). Posterior is to the left. FIGS. 17A′-G′ are high magnification of the posterior end of the eye of FIGS. 17A-G respectively (692×). Posterior eye defect area is marked with a representative dashed line. FIGS. 17H-I are graphs representing measurement of the posterior eye defect area (μm²) for dasatinib and ponatinib, respectively. Data represents mean±SEM. ** P<0.01; **** P<0.0001.

FIGS. 18 A, A′, B, B′, C, C′, D, D′, E, E′, F, F′, G, G′, H, H′, I, I′, J, J′, K, K′, L, L′ show how nilotinib shows a tendency to decrease BCR-ABL1^{p210} mediated eye defect and are scanning electron micrographs (FIGS. 18A-A′, L-L′) of adult Drosophila compound eyes from flies fed on DMSO only (FIGS. 18A-A′, D-D′, G-G′, J-J′) or nilotinib (FIGS. 18B-B′-C-C′, E-E′-F-F′, FIGS. 18HH′-I-I′, K-K′-L-L′). Posterior is to the left. GMR-GAL4>w1118 were used as control. A′-L′ are high magnification of the posterior end of the eye in A-L respectively (692×). Normal development in control flies fed on DMSO (FIGS. 18A, A′-G, G′) or nilotinib (FIGS. 18B-B′-C-C′, H-H′-I-I′) is observed. BCR-ABL1^{p210} (FIGS. 18D-D′) and BCR-ABL1^{p210/T315I} (FIGS. 18J-J′) expressing flies fed on DMSO show characteristic defective area with loss of ommatidial facets. Area is marked with a representative dashed line. Feeding low or high dose nilotinib to BCR-ABL1^{p210} (FIGS. 18E-E′-F-F′) and BCR-ABL1^{p210/T315I} (FIGS. 18K-K′-L-L′) retained the defective area in the posterior end of the eye marked with a dashed line. Compare to FIGS. 18D-D′ and FIGS. 18J-J′ respectively. FIG. 18M is a graph representing measurement of the posterior eye defect area (μm²). Data represents mean±SEM. ****, P<0.0001.

FIG. 19 is a schematic showing the procedure followed for lethality assay. Embryos expressing BCR-ABL1^{p210} or BCR-ABL1^{p210/T315I} under the control of engrailed-GAL4 are transferred from grape juice plates to the surface of food mixed with either DMSO or drug and then allowed to reach the desired developmental stage. (Created with Biorender.com)

FIGS. 20A-B show the rescue of BCR-ABL1^{p210} mediated developmental block at pupal stage by dasatinib and ponatinib, where FIG. 20A is a schematic of the quantification of rescue was based on the number of embryos (n) that survived as pharate adults (x) and eclosed adults (y). FIG. 20B is a graph with the left axis shows percent viability of control pharate and eclosed adult flies and of pharate (x/n) or eclosed (y/n) untreated (DMSO) or treated (20 μM dasatinib and 100 μM ponatinib) BCR-ABL1^{p210} expressing flies. Right axis shows percent viability normalized to percent of control adults. Total n of 200 embryos per condition.

FIGS. 21A-B shows the partial reversal of BCR-ABL1^{p210/T315I} mediated developmental block at larval stage by ponatinib, where FIG. 21A is a schematic showing the Quantification of the suppression was based on the number of embryos (n) that survived as pupae (x). FIG. 21B is a graph with the left axis shows percent viability of control pupae formed and of untreated (DMSO) or treated (100 μM ponatinib) BCR-ABL1^{p210/T315I} expressing flies. Right axis shows percent viability normalized to percent of control pupae. Total n of 200 embryos per condition.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerical reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Disclosed herein are compositions useful in an in vivo method of screening for a therapeutic for CML. Disclosed herein are compositions useful in methods of administering a candidate therapeutic for CML to Drosophila larvae. Disclosed herein are compositions useful in a method of determining the survival of the larvae to a further developmental stage when administered a candidate therapeutic. Disclosed herein are compositions useful in developing, creating, and utilizing transgenic Drosophila. For example, disclosed herein are transgenes used in Drosophila. In an aspect, a transgenic Drosophila can comprise a human BCR-ABL gene. In an aspect, a transgenic Drosophila can comprise a human BCR-ABL gene comprising one or more mutations. In an aspect, a transgenic Drosophila can comprise a human BCR-ABL gene comprising one or more substitutions. In an aspect, one or more mutations of a human BCR-ABL transgene can correspond to one or more amino acid substitution.

Disclosed herein are methods for screening for a therapeutic for CML. In an aspect, disclosed is an in vivo method of screening for a therapeutic for CML, the method comprising administering a candidate therapeutic for CML to Drosophila larvae, wherein the larvae express a human BCR-ABL transgene, and determining the survival of the larvae to a further developmental stage, wherein the survival of the larvae indicates that the candidate therapeutic for CML is a therapeutic for CML. In an aspect, the larvae can express a human BCR-ABL transgene in one or more-imaginal disc. In an aspect, a human BCR-ABL transgene can comprise one or more substitutions. In an aspect, a human BCR-ABL transgene can comprise one or more mutations. In an aspect, one or more mutations in a human BCR-ABL transgene can correspond to one or more amino acid substitution.

Disclosed are in vivo methods of screening for a therapeutic for CML, the method comprising administering a candidate therapeutic for CML, to Drosophila larvae, wherein the larvae express a human BCR-ABL transgene; and assaying for amelioration of one or more sign or symptom associated with CML, wherein the amelioration of one or more sign or symptom indicates that the candidate therapeutic for CML is a therapeutic for CML.

Assaying for amelioration of one or more sign or symptom associated with CML can involve assaying for the sign or symptom prior to administration of a candidate therapeutic and assaying for amelioration of the sign or symptom after administration of a candidate therapeutic, wherein amelioration of the sign or symptom after administration compared to the sign or symptom prior to administration indicates that the candidate therapeutic for CML is a therapeutic for CML.

Signs or symptoms associated with CML can be phenotypic or genotypic. In some aspects, phenotypic signs or symptoms can include, but are not limited to the: fatality of the larvae or pupae, ommatidial fusions, misplaced mechanosensory bristles, and a characteristic groove at the lower end of the eye characterized by the loss of ommatidial facets. Amelioration, or a decrease, in ommatidial fusions, misplaced mechanosensory bristles, and disappearance of the characteristic groove at the lower end of the eye characterized by the loss of ommatidial facets can indicate that a candidate therapeutic for CML is a therapeutic for CML. Therefore, disclosed are in vivo methods of screening for a therapeutic for CML to Drosophila larvae, wherein the larvae express a human BCR-ABL transgene; and assaying for the presence of an eye groove or larval/pupal lethality, wherein the disappearance of the eye groove and/or reversal of larval/pupal lethality indicates that the candidate therapeutic for CML is a therapeutic for CML.

Disclosed are in vivo methods of screening for a therapeutic for CML, the method comprising administering a candidate therapeutic for CML to Drosophila larvae, wherein the larvae express a human BCR-ABL transgene; and assaying for amelioration of one or more sign or symptom associated with CML, wherein the amelioration of one or more sign or symptom indicates that the candidate therapeutic for CML is a therapeutic for CML, wherein assaying for amelioration of one or more sign or symptom associated with CML comprises examining eye phenotype for presence of an eye groove, larval/pupal lethality or additional phenotypic changes that potentially could be screened in other tissues including haemocytes or a combination thereof.

Disclosed herein are therapeutics for CML. In an aspect, a disclosed therapeutic can ameliorate one or more signs or symptoms associated with CML. In an aspect, one or more signs or symptoms associated with CML can comprise phenotypic signs or symptoms. In an aspect, a disclosed method for screening for a therapeutic for CML can comprise one of the following: examining fatality of the larvae/pupae, presence of ommatidial fusions, misplaced mechanosensory bristles, and a characteristic groove at the lower end of the eye characterized by the loss of ommatidial facets. In an aspect, a disclosed method for screening for a therapeutic for CML can comprise a combination of the following: examining fatality of the larvae/pupae, presence of ommatidial fusions, misplaced mechanosensory bristles, and a characteristic groove at the lower end of the eye characterized by the loss of ommatidial facets.

Disclosed herein are Drosophila models of CML. In an aspect, Drosophila models of CML comprise a human BCR-ABL gene. In an aspect of the disclosed Drosophila models of CML comprising a human BCR-ABL gene, BCR-ABL can exhibit functional interactions with one or more components of serine/threonine kinase activity and is a guanine nucleotide exchange factor for Rho family GTPases including RhoA. The breakpoint cluster region protein (BCR) also known as renal carcinoma antigen NY-REN-26 is a protein that in humans is encoded by the BCR gene. BCR is one of the two genes in the BCR-ABL complex, which is associated with the Philadelphia chromosome. Two transcript variants encoding different isoforms have been found for this gene. BCR-ABL oncogene exists in three forms (P190, P210, and P230) but the hallmark of CML is P210: BCR-ABL exhibits the following signaling pathway: Chronic Myeloid Leukemia (CML) is a myeloproliferative neoplasm of Hematopoietic Stem Cells (HSCs) driven by a t (9; 22) (q34; q11) reciprocal chromosomal translocation shaping the Philadelphia chromosome (Ph). Fusion of the breakpoint cluster region (BCR) on chromosome 22 with the Abelson murine leukemia viral oncogene homolog 1 (ABL) tyrosine kinase of chromosome 9, results in the fusion gene BCR-ABL. This oncogene encodes a constitutively active tyrosine kinase (BCR-ABL) which leads to altered cellular survival, proliferation, differentiation and adhesion properties. More than 90% of CML patients harbor the 210-KDa BCR-ABL protein isoform (1, 2). The tyrosine kinase activity of ABL is constitutively activated when fused to BCR which leads to dimerization/tetramerization followed by autophosphorylation hence increasing the number of phosphotyrosine residues which serve as docking sites for proteins exhibiting SH2 domains. Examples of signaling pathways that are deregulated include: Ras-mitogen-activated protein kinase (MAPK) which imparts an increase in proliferation, the Janus-activated kinase (JAK)-STAT pathway which alters the transcriptional activity, and the phosphoinositide 3-kinase (PI3K)/AKT pathway which causes a decrease in apoptosis. A tyrosine phosphorylated site at the aminoterminal BCR-encoded sequences in BCR-ABL can bind to GRB2 SH2 domain and lead to a BCR-ABL-induced activation of Ras. The constitutive activation of the Ras signaling cascade culminates in the activation of mitogen-activated protein (MAP) extracellular signal-regulated kinase (ERK) ½ (MEK) as well as MAP kinase proteins. Regulation of apoptosis is one of the means by which BCR-ABL mediates its oncogenic effects and PI3K activation leads to the activation of AKT kinase which in its turn phosphorylates players in apoptosis regulation leading to increased survival of the abnormal clone (reviewed in Cilloni D, Saglio G. Molecular pathways: Bcr-abl. Clinical Cancer Research. 2011 Dec. 8:clincanres-1613). TKIs competitively inhibit the Adenosine triphosphate (ATP) binding site in the BCR-ABL kinase domain and hence block the phosphorylation of proteins which play a role in BCR-ABL signal transduction cascade; culminating in apoptosis and inhibition of proliferation of CML cells (3).

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

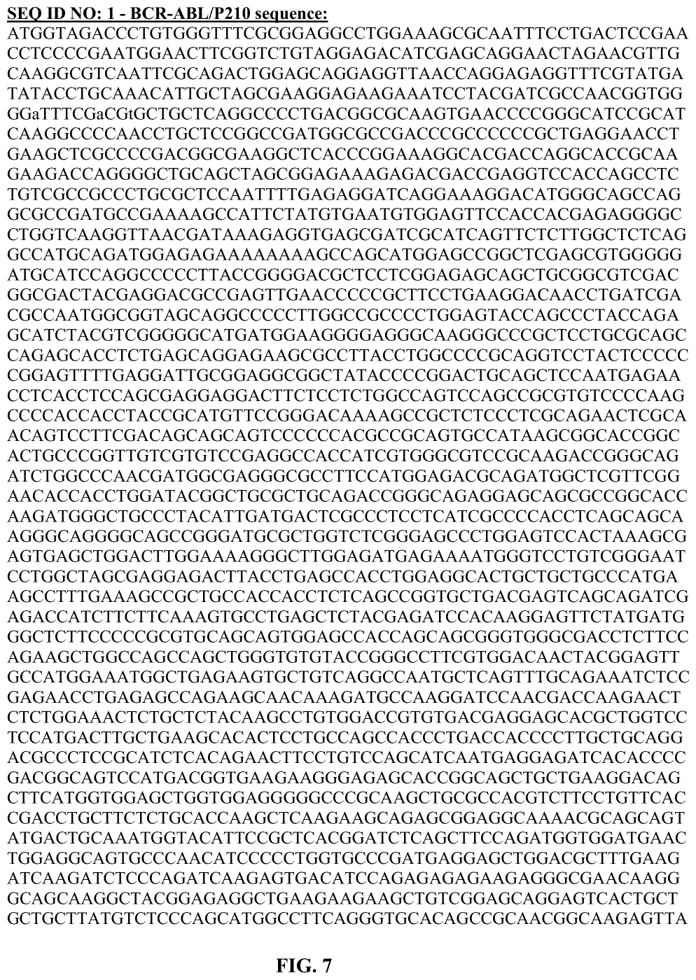

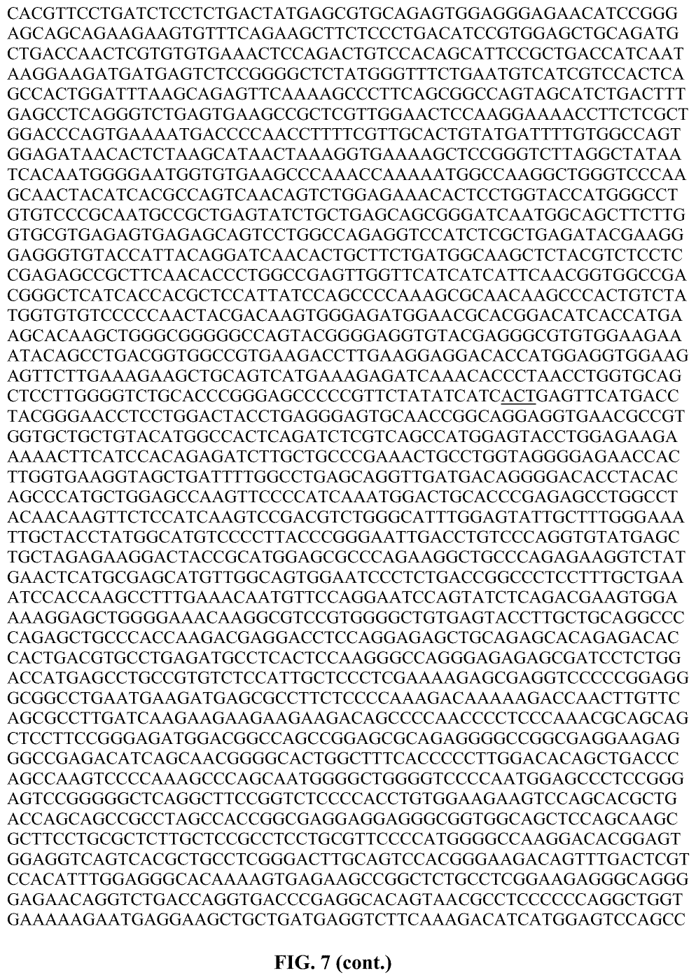

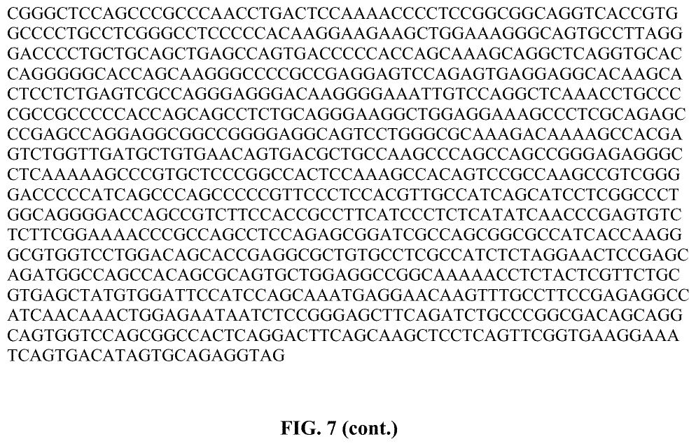

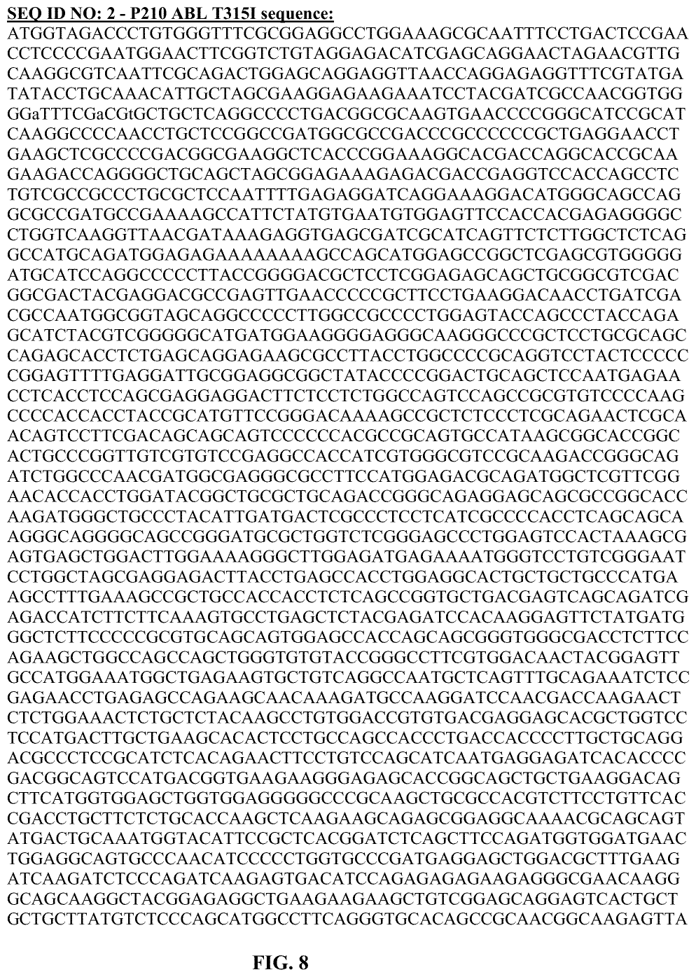

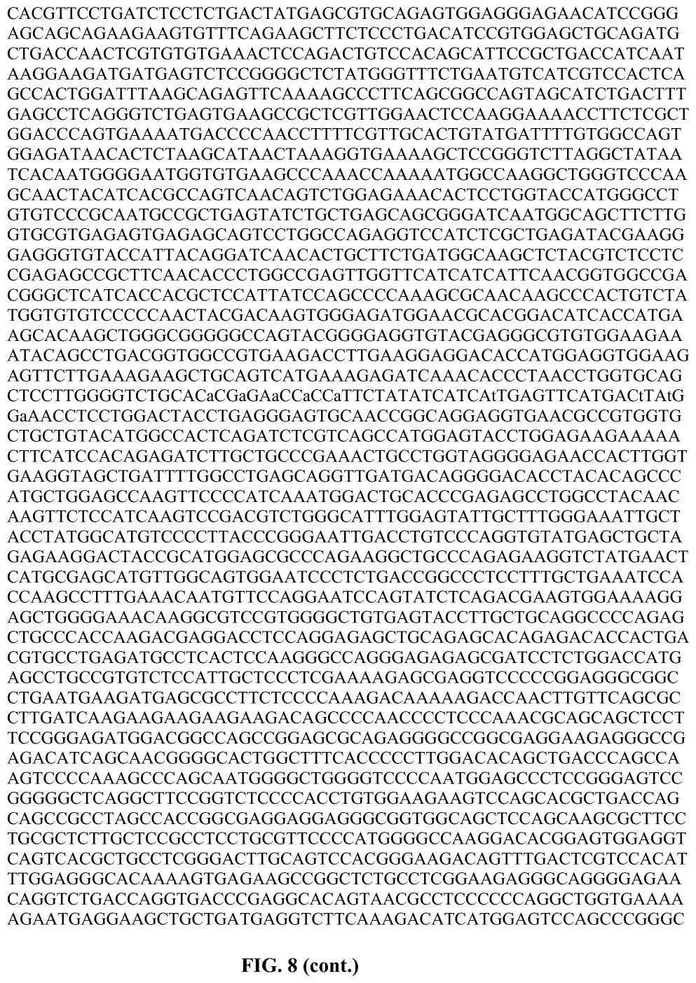

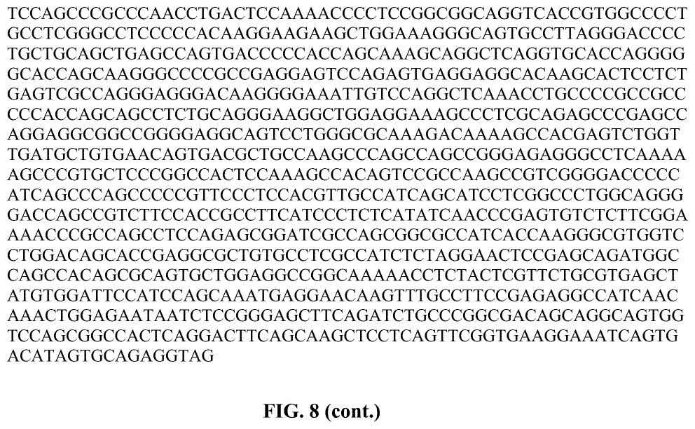

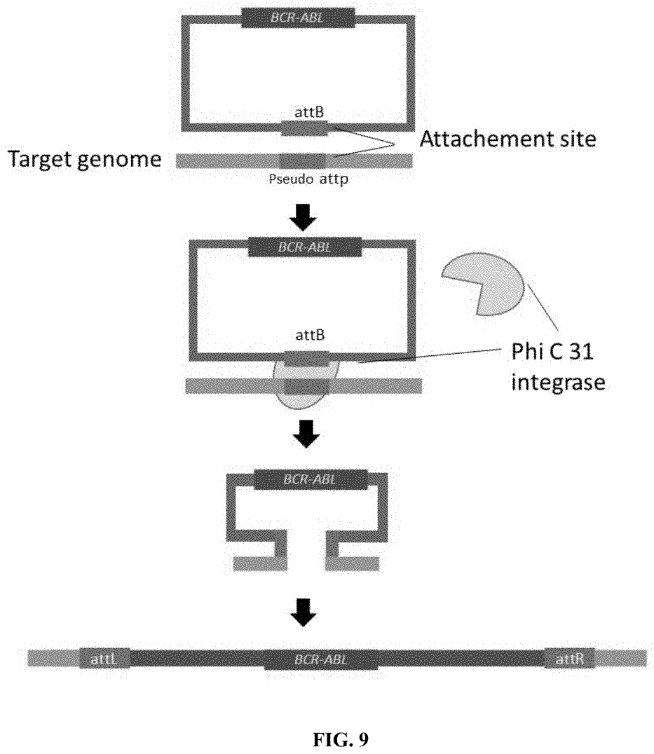

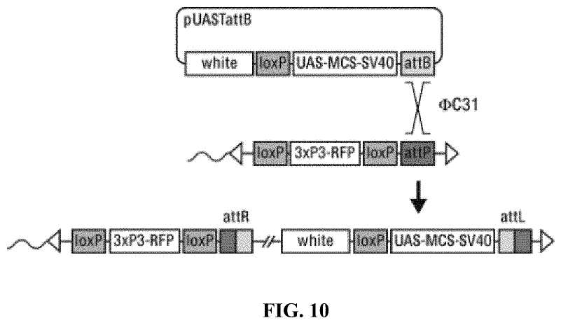

In one embodiment, the method disclosed herein allows for rapid and efficient in vivo screening of treatments for CML using the fruit fly (Drosophila melanogaster). Transgenic flies, harboring the human BCR-ABL (SEQ ID NO: 1 is wild-type p210 and SEQ ID NO: 2 is p210^{T315I}), as shown in FIGS. 7-8, were generated using the Phi C31 integrase system, as shown in FIG. 9, which is a site-specific integration system, and were inserted on the 3rd chromosome for GAL4-UAS expression. Myc tag was added at N-terminus to identify expression of the BCR-ABL protein. BCR-ABL (wild-type p210 and p210^{T315I}) were inserted into pUAST-attB Drosophila expression vector, as shown in FIG. 10, (http://flyc31.org/sequences_and_vectors.php. The sequence of the pUASTattB vector can be found in the GenBank data base under the accession number EF362409) at EcoRI-(Spel-xbal) site. pUAST-attB-myc BCR-ABL wild-type and pUAST-attB-myc BCR-ABL T315I were injected into y1 w67c23; P{CaryP} ABLattP2 (8622 BDSC) embryos in order to generate transgenic flies.

PhiC31 integrase-mediated transgenesis systems are based on the site-specific bacteriophage PhiC31 integrase which mediates sequence-directed, irreversible and highly efficient integration between a bacterial attachment site (attB) and a phage attachment site (attP). Injecting plasmid containing attB site and white marker into attP-containing docking site strain(s) with PhiC31 activity makes the resultant stable w+ transformants containing your gene-of-interest between attL and attR sites (irreversible).

With the PhiC31 Integrase System, the gene-of-interest is cloned into the attB Donor Plasmid, and then (Step 1) co-transfect with a PhiC31 Integrase Expression Plasmid. (Inside the cell) The PhiC31 Integrase is transiently expressed, and mediates site-specific recombination between the attB site on the donor plasmid and a pseudo attP site in the genome. Because pseudo attP sites are typically present in transcriptionally active sites of the genome, your gene-of-interest will likely be integrated into an active region of the genome.

Alternative methods using different drivers may be employed. In one embodiment, driving the expression of BCR-ABL in Drosophila imaginal discs is by using engrailed-GAL4 and in Drosophila eyes using Glass multimer reporter (GMR)-GAL4 however other drivers such as those that direct the expression in epithelial tissues and hematopoietic compartments can be also used. In one embodiment, the method of lethality phenotype reversal is one approach that is more efficient for drug screening. GAL4-UAS system was used to drive the expression of BCR-ABL (wild-type p210 and p210^{T315I}) in Drosophila posterior compartments of imaginal discs using engrailed-GAL4. In another embodiment, GMR-GAL4 may be used to drive the expression of BCR-ABL (wild-type p210 and p210^{T315I}) in Drosophila eye discs.

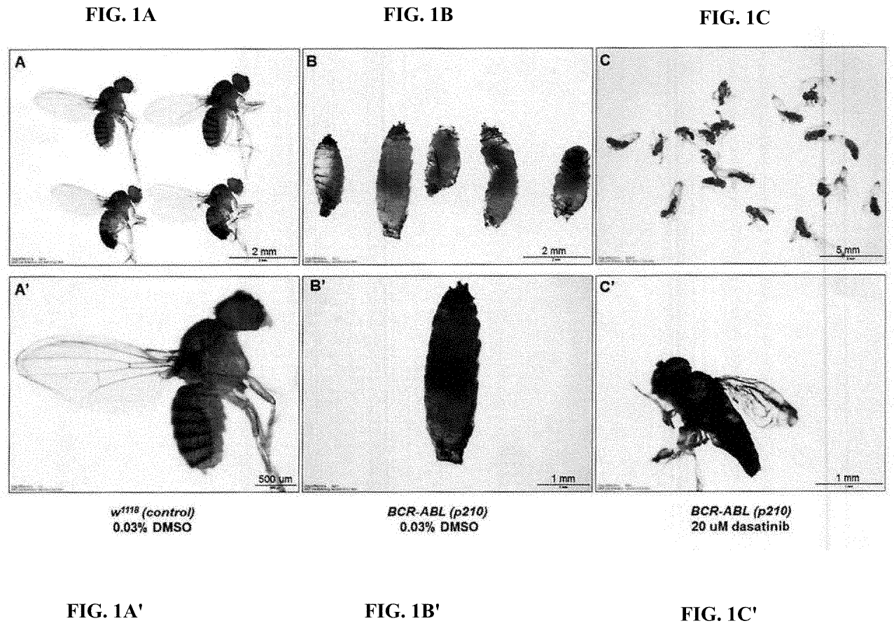

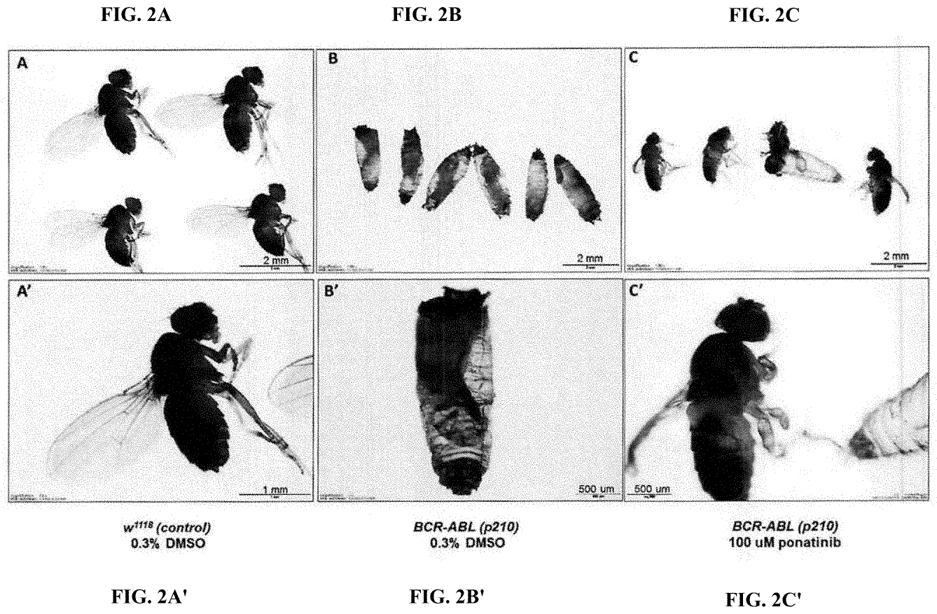

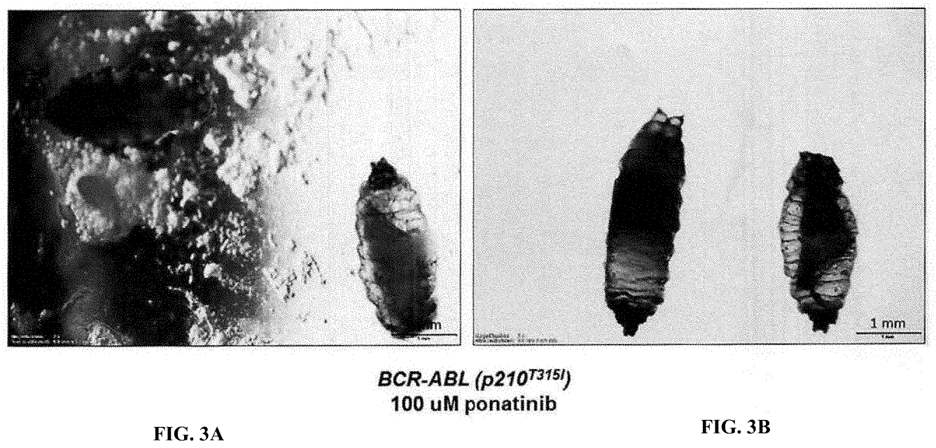

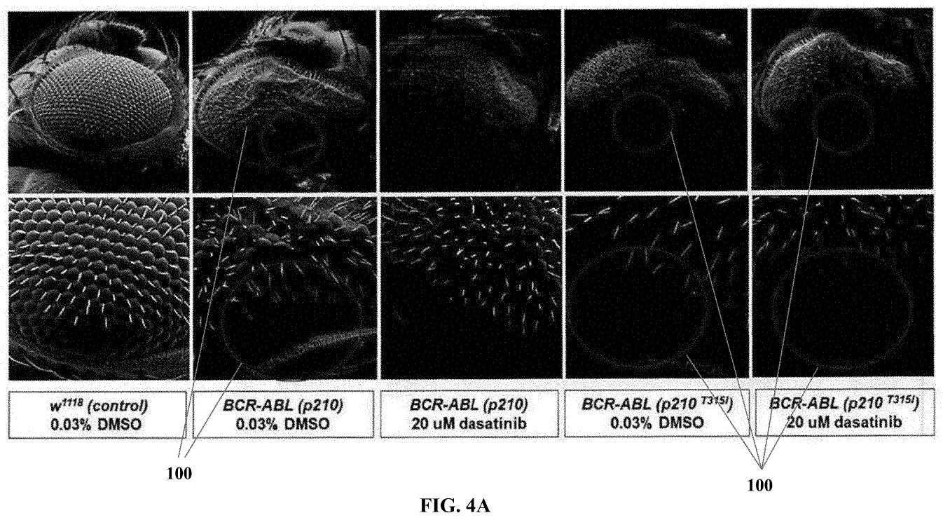

In one embodiment, a lethality phenotype reversal model is used and comprises: Flies carrying human BCR-ABL (wild-type p210 and p210^{T315I}) were crossed to engrailed-GAL4 flies and cultured at 25° C. on food only or food mixed with DMSO or tyrosine kinase inhibitors (dasatinib and ponatinib). The results showed that progeny which were cultured on food only or food mixed with DMSO expressing BCR-ABL (wild-type p210) and BCR-ABL (p210^{T315I}) showed pupal lethality (FIG. 1) and embryonic/larval lethality respectively. Progeny expressing BCR-ABL (wild-type p210) which were cultured on dasatinib (20 uM) (FIG. 1) and ponatinib (100 uM) (FIG. 2) showed a reversal of pupal lethality and enclosure of adult flies. Progeny expressing BCR-ABL (p210^{T315I}) which were cultured on food containing ponatinib (100 uM) showed evidence of reversal of larval lethality (pupal development) (FIG. 3).

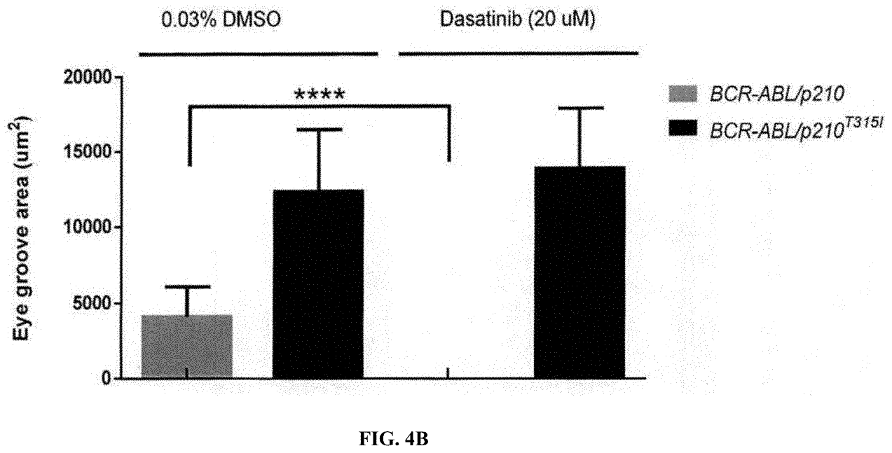

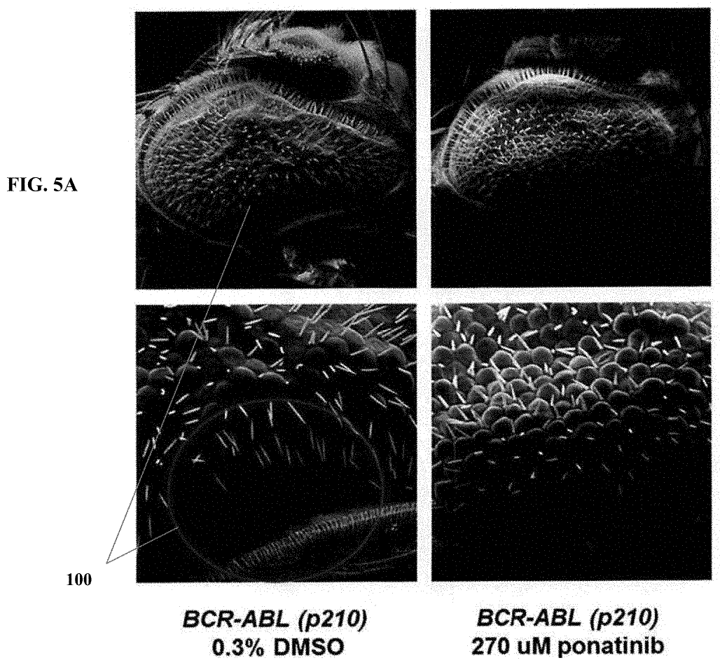

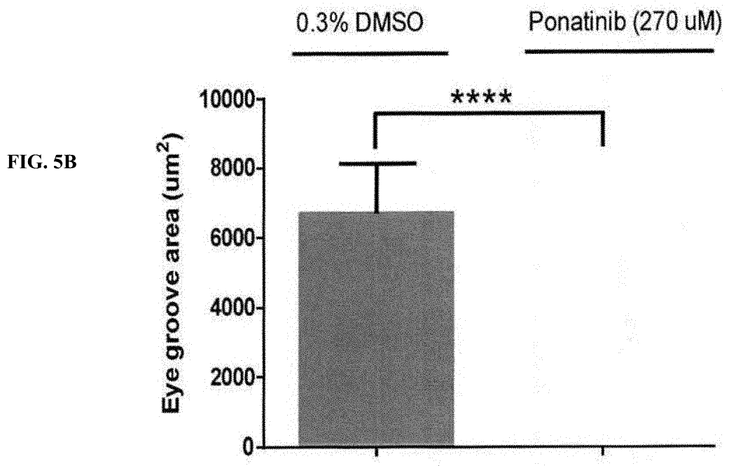

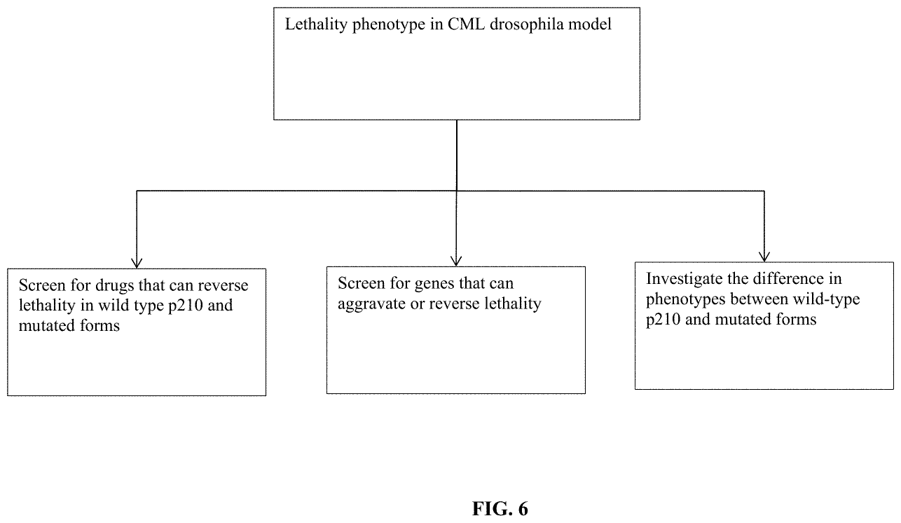

The method of rough eye phenotype reversal comprises: Flies carrying human BCR-ABL (wild-type p210 and p210^{T315I}) were crossed to GMR-GAL4 flies and cultured at 18° C. on food only or food mixed with DMSO or TKIs (dasatinib and ponatinib). After enclosure of adult flies; they were fixed with a mixture of glutaraldehyde and formaldehyde and dehydrated by increasing ethanol concentrations (30%, 50%, 70%, 80%, 90% and twice in 100%) then samples were dried using a critical point dryer and coated with a 20 nm layer of gold and visualized using a Tescan, Mira III LMU, FEG (scanning electron microscope) Field Emission Gun and secondary electron detector. Progeny expressing BCR-ABL (wild-type p210 and p210^{T315I}) that were cultured on food only or food mixed with DMSO developed a rough eye phenotype characterized by ommatidial fusions, misplaced mechanosensory bristles and a characteristic area at the lower end of the eye characterized by loss of ommatidial facets; which is named eye groove. The results showed that progeny expressing BCR-ABL (wild type p210) which were cultured on dasatinib (20 uM) and ponatinib (270 uM) showed a reversal of the rough eye phenotype characterized by the disappearance of the eye groove and restoration of ommatidial development in this area (FIGS. 4 and 5). DMSO was used at a concentration of 0.03% and 0.3%. Image J was used to measure the contour area of the eye groove in um² (FIGS. 4B and 5B). Statistical analyses were done by one-way analysis of variance using GraphPad Prism 6. At least 20 flies were assayed from each genotype except for BCR-ABL (p210) group on 270 uM ponatinib whereby 11 flies were assayed. As expected, T315I mutation is resistant to dasatinib and this was clearly shown in the results whereby progeny expressing BCR-ABL (p210^{T315I}) which were cultured on dasatinib (20 uM) revealed no disappearance of the eye groove area (FIG. 4A). In all experiments, the control groups were w¹¹¹⁸ flies crossed to the GAL4 lines. Further experiments may include an additional number of flies.

A tyrosine kinase inhibitor (TKI) is a pharmaceutical drug that inhibits tyrosine kinases. Tyrosine kinases are enzymes responsible for the activation of many proteins by signal transduction cascades. The proteins are activated by adding a phosphate group to the protein (phosphorylation), a step that TKIs inhibit. TKIs are typically used as anticancer drugs. For example, they have substantially improved outcomes in CML.

Ponatinib is a third generation multi-targeted tyrosine-kinase inhibitor. The primary target for ponatinib is BCR-ABL, an abnormal tyrosine kinase that is the hallmark of CML and Ph+ ALL. CML is characterized by an excessive and unregulated production of white blood cells by the bone marrow due to a genetic abnormality that produces the BCR-ABL protein. Dasatinib is a targeted therapy and a second generation tyrosine-kinase inhibitor. The main targets of dasatinib are BCR/ABL (the “Philadelphia chromosome”), Src, c-Kit, ephrin receptors, and several other tyrosine kinases.

The one embodiment using engrailed-GAL4 (lethality phenotype) can be efficiently used to screen for drugs that can interfere with BCR-ABL induced phenotypes. High throughput Screening is further described below. The embodiment using the engrailed-GAL4 (lethality phenotype) is a rapid and easy method that saves a lot of time and efforts whilst testing a large number of drugs in an in vivo context. For example a single experiment using engrailed GAL4 at 25° C. in Drosophila for detecting whether a drug can rescue lethality can give results within 12 days in contrast to 6-8 weeks in the mouse model. The eye phenotype method can be coupled to the lethality phenotype method for drugs that might not be recognized as targets in the latter.

The significance of this discovery is in its potential to refine drug discovery in CML field which warrants further research to provide better drug alternatives that can eradicate the disease. Hereby, the disclosed method and approach can be used to screen for treatments for the famous elusive T315I mutation, single and compound mutations in CML patients as well as for potential candidate drugs that can target CML stem cells. In other words, the described Drosophila CML in vivo model and system is efficiently used in the field of CML drug discovery for tackling major treatment obstacles. This model can also be used for genetic screening which could lead to deciphering important genetic players that can constitute future drug targets.

In comparison to in vivo mouse models, in vitro and in silico CML drug discovery tools, the above described in vivo Drosophila CML model can be more descriptive of the complexity of organismal response to drugs whilst providing a fast and efficient platform for high throughput screening. In vitro and in silico CML tools lack the integrity of the in vivo microenvironment and mouse models cannot be used to test a large number of drugs which can be easily made in flies, due to the rapid generation time of Drosophila, the large brood size and the ease of their handling. The system for screening can filter from a library of compounds the drug targets that are most likely to be potential CML treatments and then the targets can be tested/validated in other in vitro/in vivo approaches, therefore, speeding up the drug discovery process. For all the advantages of Drosophila in vivo models mentioned earlier, the CML field is in need of a Drosophila CML model and is established herein.

The data from the expression of BCR-ABL and BCR-ABL (p210^{T315I}) in imaginal discs show a response to both dasatinib and ponatinib treatment in the BCR-ABL (wild-type p210) flies and to ponatinib in BCR-ABL (p210^{T315I}).

Validation of a Drosophila Model of Wild Type and T315I Mutated BCR-ABL1 in Chronic Myeloid Leukemia: An Effective Platform for Treatment Screening

In this embodiment, human BCR-ABL1^{p210} and mutated BCRABL1^{p210/T315I} in Drosophila compound eyes is overexpressed. BCR-ABL1^{p210/T315I} expression induced significantly more severe rough eye phenotype compared to BCR-ABL1^{p210} pointing towards more aggressive tumorigenic capacities of the gate keeping mutation. The system comprises the efficiency of the current TKIs used in clinics in modifying the characteristic eye phenotypes of transgenic flies. Dasatinib and ponatinib rescued the eye defects observed upon expression of BCR-ABL1^{p210} making this system a valuable screening platform to pre-clinically evaluate the efficacy of potential novel therapies for CML.

Methods:

Fly stocks were maintained at about 25° C. on standard agar-based medium. GMR-GAL4 (BDSC 1104) were obtained from Bloomington Stock Center. Treatment was performed at 18° C. Fly work was done following institutional guide for the care and use of laboratory animals.

Transgenic flies, harboring human BCR-ABL1^{p210} and BCR-ABL1^{p210/T315I} were generated using Phi C31 integrase system and were inserted on the 3rd chromosome for GAL4-UAS expression. BCR-ABL1^{p210} and BCR-ABL1^{p210/T315I} were inserted into pUAST-attB Drosophila expression vector (Custom DNA cloning). pUAST-attB-myc BCR-ABL1^{p210} and pUAST-attB-myc BCR-ABL1^{p210/T315I} were injected into y1 w67c23; P{CaryP} ABLattP2 (8622 BDSC) embryos to generate transgenic flies (BestGene Inc, Chino Hills, Calif.).

Imatinib (I-5577), nilotinib (N-8207), dasatinib (D-3307) and ponatinib (P-7022) were obtained from LC laboratories, MA, USA. Stock solutions were dissolved in DMSO and the required amount of TKI was added to instant Drosophila medium (Carolina Biological Supply Company). Since DMSO is known to be toxic to Drosophila⁴⁰, 0.03% DMSO was used for low TKI concentrations and 0.3% for high concentrations.

Scoring of eye phenotypes and measurement of eye defect area

A grading score, that was modified from the score previously published³⁵, was used for scoring and is based on the number of ommatidial fusions, the extent of bristle organization and ommatidial loss (Table 1). For measurement of the posterior eye defect area, Image J 36 was used. SEM images were coded by one researcher and analysis was blindly performed by another researcher. n=20 flies from each genotype at each temperature was scored and the experiment was done in triplicate. For measurement of posterior eye defect area an average of n=20-30 flies from each group was quantified and the experiment was done at least two times.

TABLE 1
Grading score for quantification of eye roughness.

| Score | Criteria |
|---|---|
| 0 | Regular ommatidial facets and bristle organization |
| 1 | Scattered areas of non-polarized bristle alignments |
| | And less than 4 scattered areas displaying fusions of |
| | ommatidial facets |
| 2 | Scattered areas of non-polarized bristle alignments |
| | And 10-20 fusions of ommatidial facets that are scattered or in the |
| | same area |
| | with/without duplicated bristles |
| | with/without few lens defects manifested as holes |
| | in the ommatidial facets |
| 3 | Scattered areas of non-polarized bristle alignments |
| | And 20-40 fusions of ommatidial facets that are scattered or in the |
| | same area |
| | with/without duplicated bristles |
| | with/without few lens defects manifested as holes |
| | in the ommatidial facets |
| 4 | One large surface area of non-polar bristle alignments and fusions |
| | of ommatidial facets of the same large area |
| | with/without duplicated bristles |
| | with/without few lens defects manifested as holes |
| | in the ommatidial facets |
| 5 | Multiple non-polar bristle alignments |
| | And one large surface area of fusions of ommatidial facets and/or |
| | duplicated bristles |
| | with/without few lens defects manifested as holes |
| | in the ommatidial facets |
| 6 | Multiple non-polar bristle alignments |
| | And scattered areas of incompletely developed ommatidial facets |
| | and/or duplicated bristles |
| | with/without lens defects manifested as holes |
| | with/without a characteristic groove of lost ommatidial |
| | facets on the lower end of the eye |
| 7 | Multiple non-polar bristle alignments |
| | And one large surface area of incompletely developed ommatidial |
| | facets and/or duplicated bristles |
| | With/without lens defects manifested as holes in the residual |
| | ommatidial facets |
| | With/without a characteristic groove of lost ommatidial facets on |
| | the lower end of the eye |
| 8 | Multiple non-polar bristle alignments |
| | And/or duplicated bristles |
| | with total loss of ommatidial facets |
| | And with/without 1 area of missing bristles |
| 9 | Multiple non-polar bristle alignments |
| | And/or duplicated bristles |
| | With total loss of ommatidial facets |
| | With more than 1 area of missing bristles |
| 10 | Few dispersed bristles across the eye with total |
| | loss of ommatidial facets |

Scanning Electron Microscopy (SEM)

Adult flies were fixed in 2% glutaraldehyde and 2% formaldehyde in phosphate buffered saline (PBS) (1×), washed, dehydrated with a series of increasing ethanol concentrations, dried with critical point dryer (k850, Quorum Technologies), mounted on standard aluminum heads and coated with 20 nm layer of gold. Analysis was performed using Tescan, Mira III LMU, Field Emission Gun (FEG) SEM with Secondary Electron detector.

Western Blot Analysis

Fly heads were homogenized in Laemmli buffer and samples were loaded in 8% SDSPAGE. Anti ABL1 (SC-23, 1:1000, Santa Cruz Biotechnology, Santa Cruz, Calif.) and phospho-ABL1 (#2868, 1:500, Cell Signaling Technology) primary antibodies and antimouse (SC-2318, 1:5000, Santa Cruz Biotechnology, Santa Cruz, Calif.) and anti-rabbit (NA934, 1:5000, GE Healthcare) secondary antibodies were used for protein detection. An extract (150 μg) from 20-30 flies was used.

Statistical Analysis

The statistical significance of difference between the average scores of rough eye phenotype and average scores of posterior eye defect area was evaluated using two-way analysis of variance (ANOVA) followed by Tukey's multiple comparisons test. One-way ANOVA was used when comparing averages of posterior eye defect area for dose response and was followed by Tukey's multiple comparisons test. Associations with p<0.05 were considered significant. Statistical tests were done using GraphPad Prism 6.0 software.

Lethality Phenotype Analysis

Engrailed-GAL4 was used to drive the expression of BCR-ABL1^{p210} and BCR-ABL1^{p210/T315I} in posterior compartments of imaginal discs. Virgin females from engrailed-GAL4 line were crossed to males from BCR-ABL1 lines. Flies from engrailed-GAL4>w¹¹¹¹⁸ were used as control. Parent flies were allowed to mate and females laid eggs on the surface of grape juice plate to which a thin layer of yeast solution was added on the surface. Then parents were discarded and a known number of embryos was transferred to a vial containing either 0.1% DMSO only or the desired concentration of drug mixed with food. Embryos were monitored for survival into the desired stage (FIG. 19). Images of pupae or adults were taken using Olympus SZX10 stereomicroscope.

Results:

Expression of Human BCR-ABL1 in Drosophila Eyes Induces Transformation

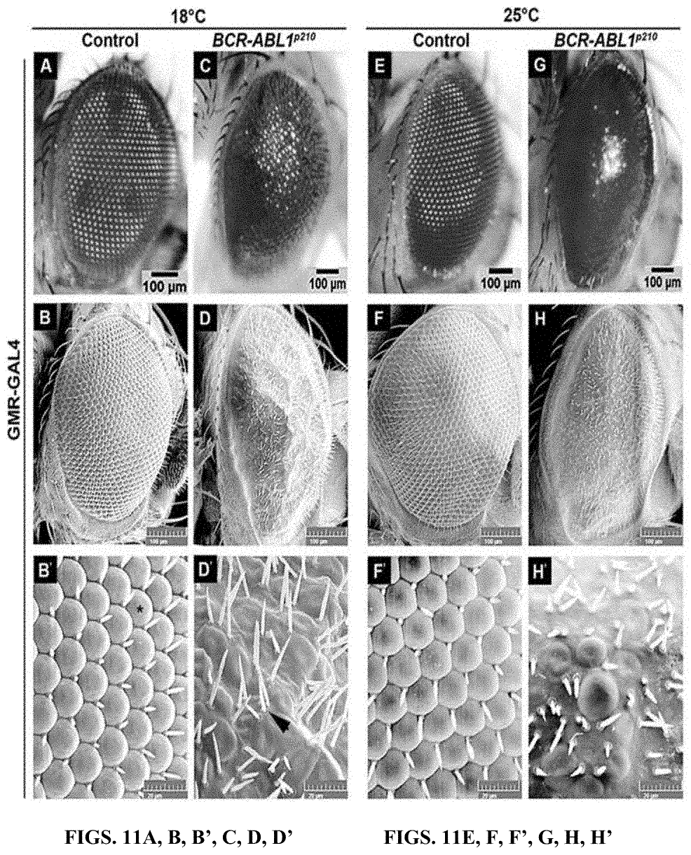

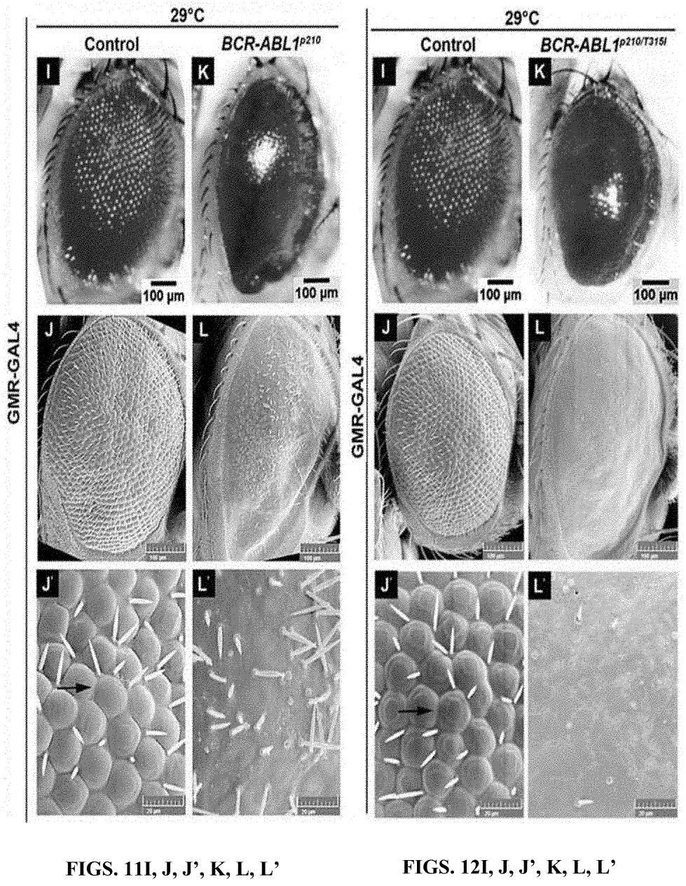

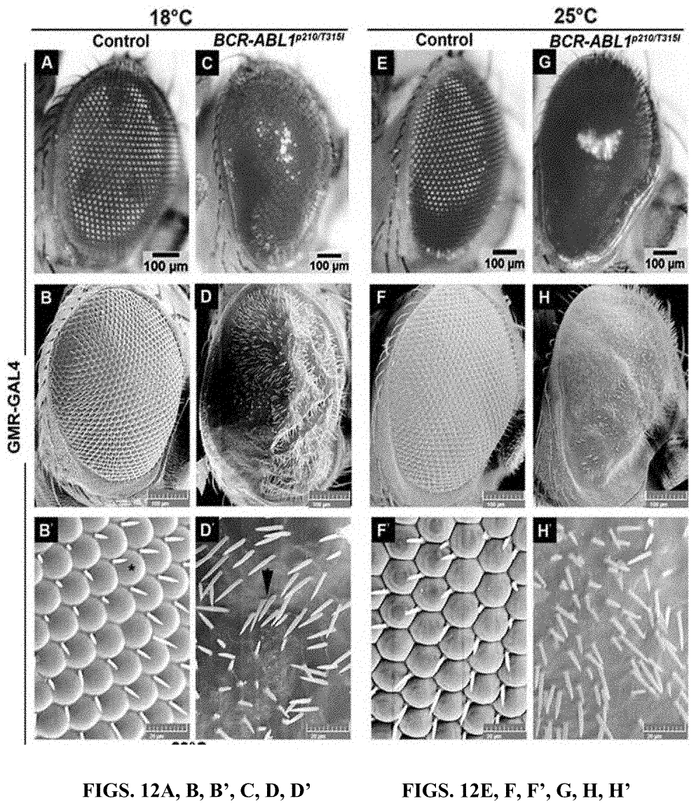

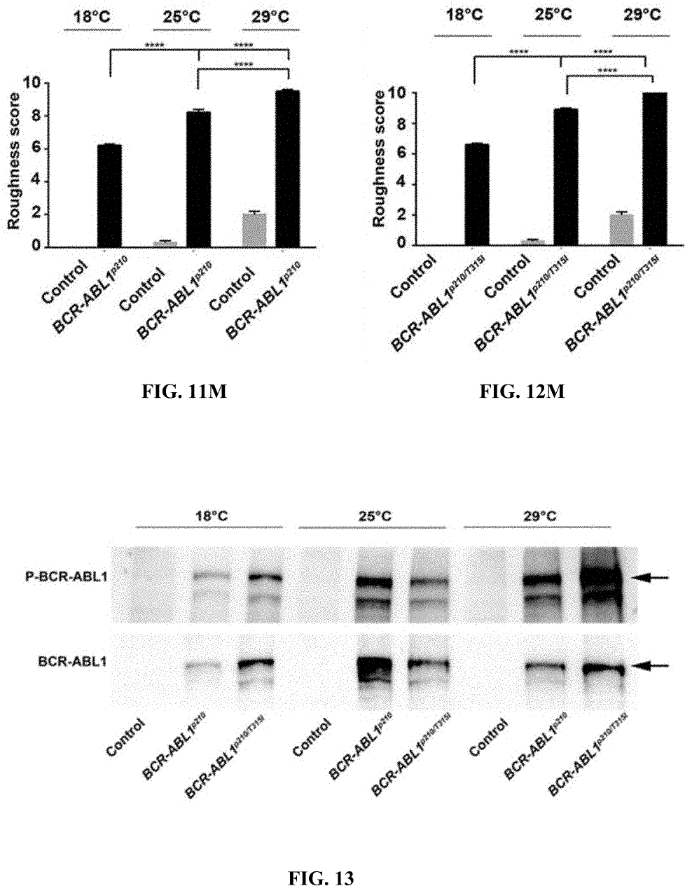

To assess the transformative potential of human BCR-ABL1^{p210} and BCR-ABL1^{p210/T315I} in Drosophila, the transgenes were expressed in the adult eye using the glass-multimer reporter promoter GMR-GAL4 which drives the expression in all differentiating photoreceptor cells posterior to the morphogenetic furrow³⁷. GMR-GAL4>w1118 flies were used as a control. That GAL4-UAS system is temperature sensitive allows control of BCR-ABL1 expression levels³⁸. Therefore; crosses were performed at 18° C., 25° C., and 29° C. allowing for a reciprocal increase in transgene expression upon increased temperatures. Eclosed flies were imaged using light microscopy and SEM and evaluations of phenotypes were performed using a grading score (Table 1) which graded the severity of the phenotype based on the extent of mechano-sensory bristles alignment, misplacement and duplication as well as the extent of ommatidial facets loss indicating disruption in cellular proliferation and differentiation collectively defining interrupted normal development³⁹. BCR-ABL1^{p210} and BCR-ABL1^{p210/T315I} showed a rough eye phenotype with increased severity at a higher temperature compared to control flies. At 18° C. BCR-ABL1^{p210} and BCR-ABL1^{p210/T315I} flies exhibited a rough eye phenotype characterized by ommatidial fusions and areas of lost ommatidial facets, particularly at the posterior end of the eye, as well as multiple ectopic mechano-sensory bristles which are duplicated at some instances (FIGS. 11C-D-D′; FIGS. 12 C-D-D′). At 25° C., a more severe rough eye was observed in both BCR-ABL1^{p210} and BCRABL1^{p210/T315I} with loss of the majority of ommatidial facets (FIGS. 11 G-H-H′; FIGS. 12 GH-H′). At 29° C., the severity increased to involve loss of the majority of mechanosensory bristles in addition to the total loss of ommatidial facets in both BCR-ABL1^{p210} and BCR-ABL1^{p210/T315I} expressing flies (FIGS. 11K-L-L′; FIG. 12 K-L-L′). The average roughness of BCR-ABL1^{p210} significantly increased from 6.2 at 18° C. to 8.2 (P<0.0001) at 25° C. and to 9.5 (P<0.0001) at 29° C. (FIG. 11M). As for BCR-ABL1^{p210/T315I}, the average roughness significantly increased from 6.6 at 18° C. to 8.9 (P<0.0001) at 25° C. and to 10 (P<0.0001) at 29° C. (FIG. 12M). Western blot analysis confirmed the expression and phosphorylation of BCR-ABL1^{p210} and BCR-ABL1^{p210/T315I} in Drosophila eyes (FIG. 13).

Dasatinib and Ponatinib Rescue Human BCR-ABL1p210 Mediated Defects in Drosophila

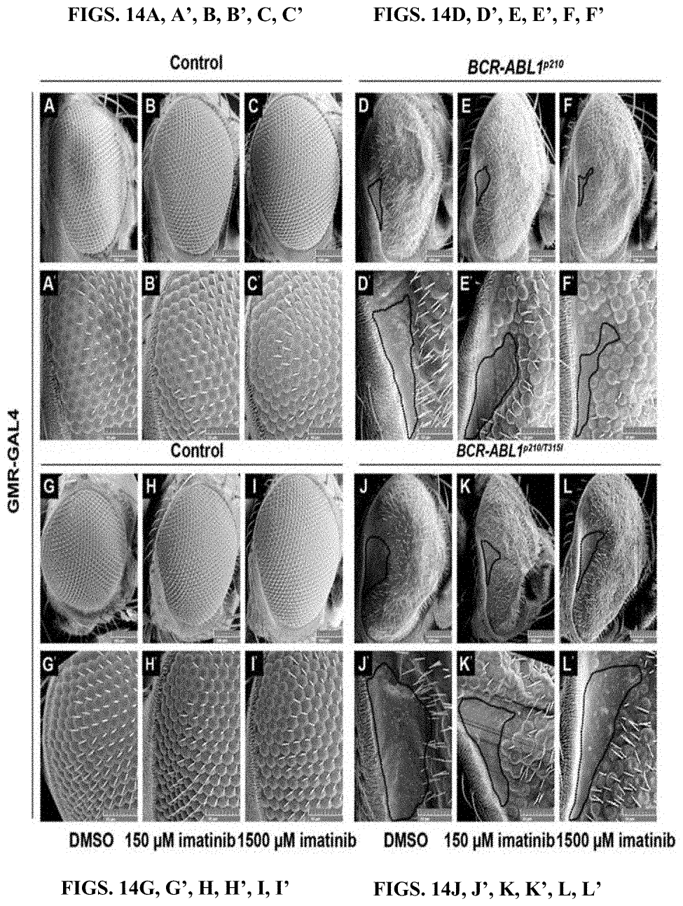

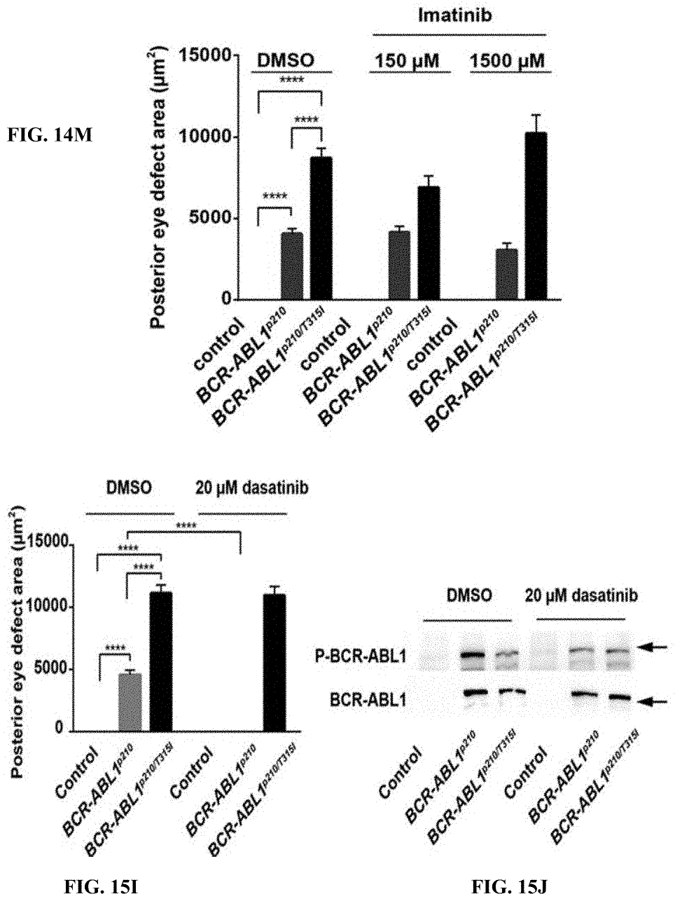

Since expression of BCR-ABL1 at high temperature induced severe eye defects in adult flies, the lowest temperature (18° C.) was used to produce milder phenotypes for TKI screening efficiency allowing for easy visualization of any rescue due to drug activity. Four TKIs were tested which included imatinib, nilotinib, dasatinib and ponatinib. BCR-ABL1^{p210} flies were crossed to GMR-Gal4 flies and progeny were fed on multiple concentrations of the TKIs (treated) or on DMSO alone (untreated). Untreated BCR-ABL1p210 and BCR-ABL1^{p210/T315I} flies showed the same defects described previously at 18° C. focusing particularly on the posterior end of the eye with a characteristic defective area characterized by loss of ommatidial facets (FIGS. 14-16). The posterior eye defect area in untreated BCR-ABL1p210 flies showed an average of 4580 μm² and 4044 μm² on 0.03% DMSO and 0.3% DMSO respectively (FIGS. 14-16).

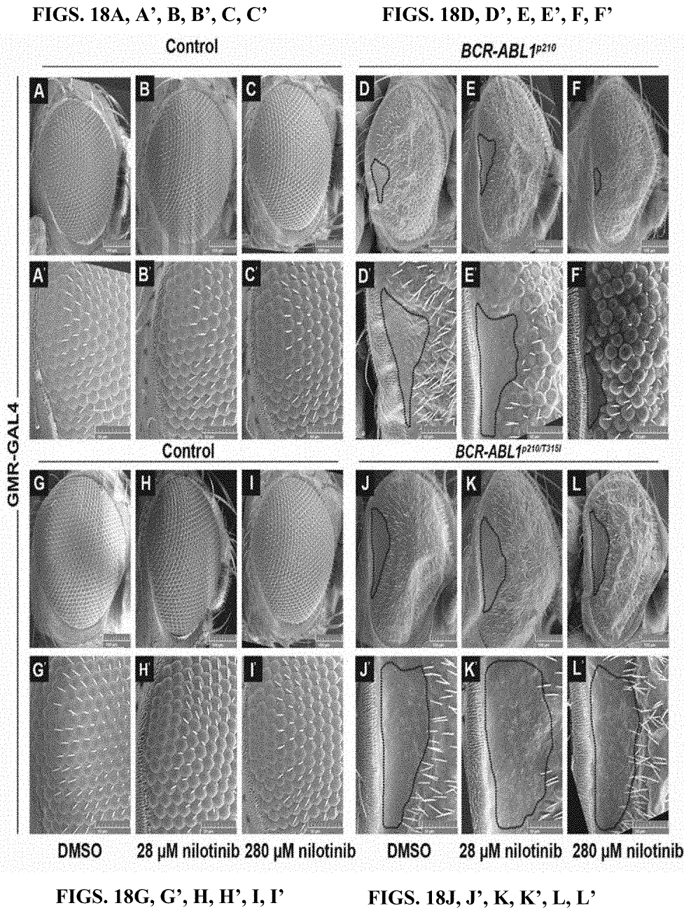

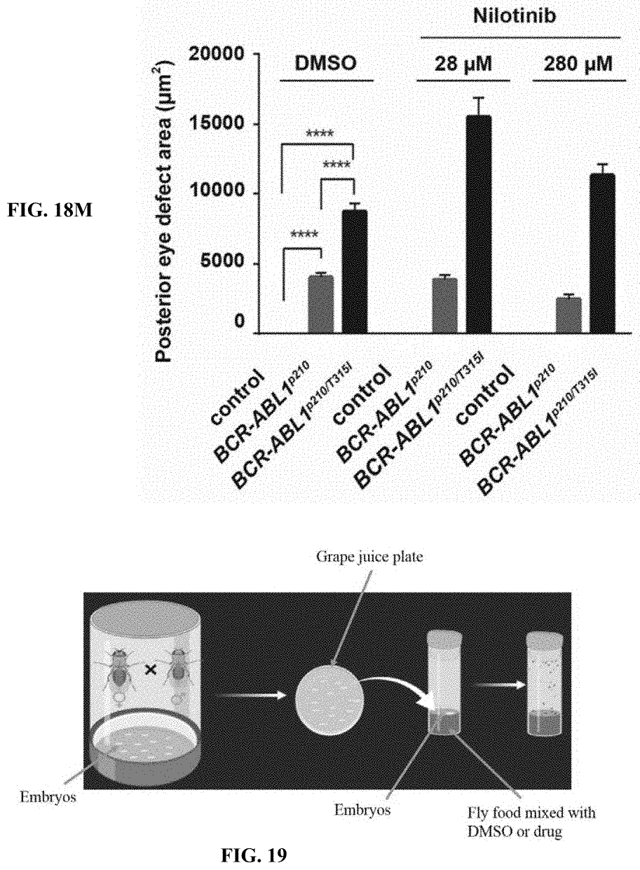

On the other hand, untreated BCR-ABL1^{p210/T315I} expressing flies showed a wider area of defect at the posterior end with an average significant increase in defect area to 11148 μm² (P<0.0001) and 8728 μm² (P<0.0001) on 0.03% DMSO and 0.3% DMSO respectively as compared to untreated BCR-ABL1^{p210} expressing flies (FIGS. 14-16). Feeding 150 μM or 1500 αM imatinib to BCR-ABL1^{p210} expressing flies did not eliminate the posterior eye defect. However, the average posterior eye defect area showed a tendency to decrease with 1500 μM imatinib (3047 μm²) as compared to that of 150 μM imatinib (4142 μm²) and untreated flies (4044 μm²) (FIG. 14M). In one embodiment, the percentage of flies with total rescue (total disappearance of the posterior eye defect) with 150 μM and 1500 μM imatinib was 4% and 21% respectively. Similarly, feeding 28 μM (FIGS. 18 E-E′) or 280 μM (FIGS. 18 F-F′) nilotinib to BCR-ABL1^{p210} expressing flies did not eliminate the posterior eye defect. However, the average posterior eye defect area showed a tendency to decrease with 280 μM nilotinib (2480 μm²) as compared to that of 28 μM nilotinib (3871 μm²) and untreated flies (4044 μm²) (FIG. μm²). The percentage of flies with total rescue with 28 μM and 280 μM nilotinib was 7% and 13% respectively.

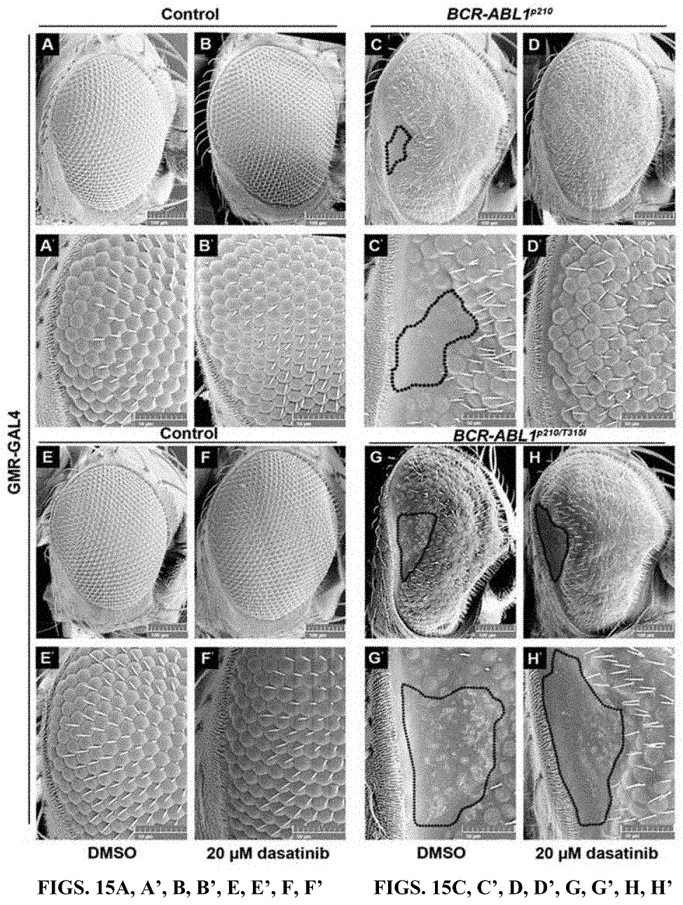

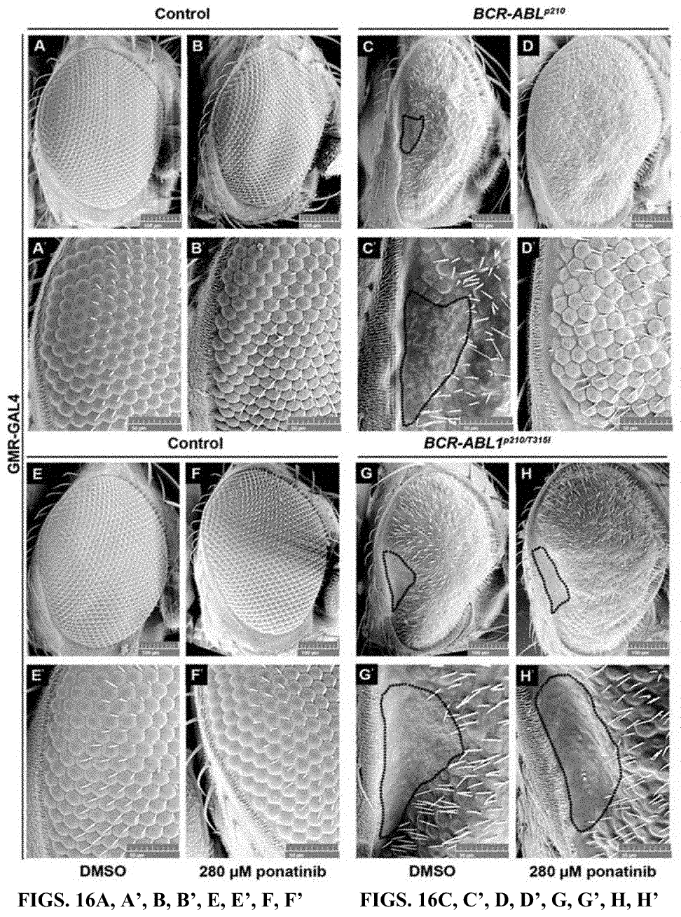

Testing the potent TKIs (dasatinib and ponatinib) showed more efficient rescue. Feeding 20 μM dasatinib or 280 μM ponatinib to BCR-ABL1^{p210} expressing flies improves the overall eye ommatidial arrangement and more specifically eliminates the characteristic posterior eye defect by restoring its normal ommatidial development (FIGS. 15 D-D′; FIGS. 16 D-D′). The average posterior eye defect area significantly decreased from 4580 μm² (in untreated flies) to 0 μm² (P<0.0001) with 20 μM dasatinib (FIGS. 1I-J) and from 4044 μm² (in untreated flies) to 267 μm² (P<0.0001) with 280 μM ponatinib (FIGS. 16I-J). The percentage of flies with total rescue was 100% with dasatinib and 86% with ponatinib.

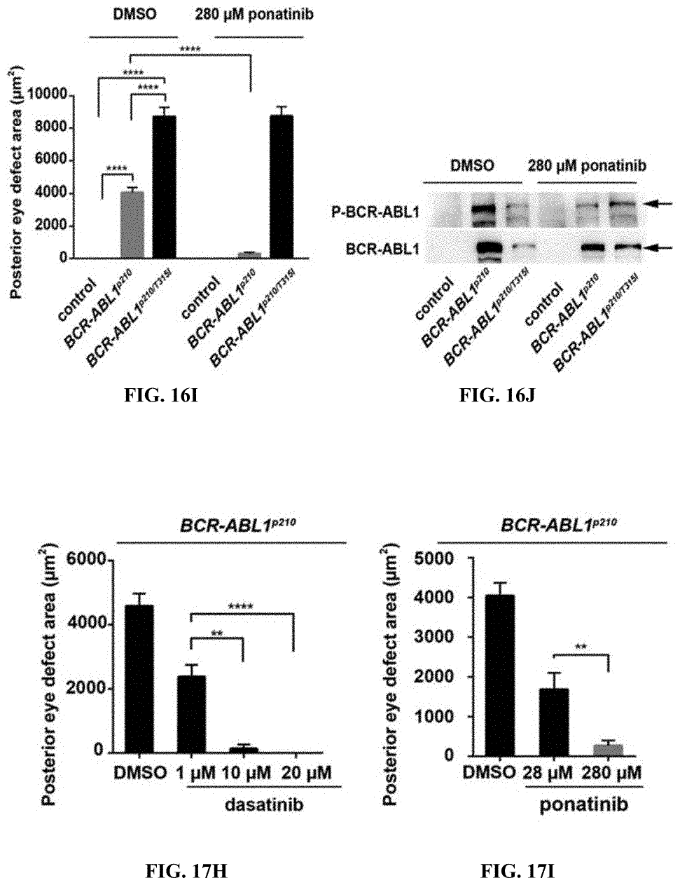

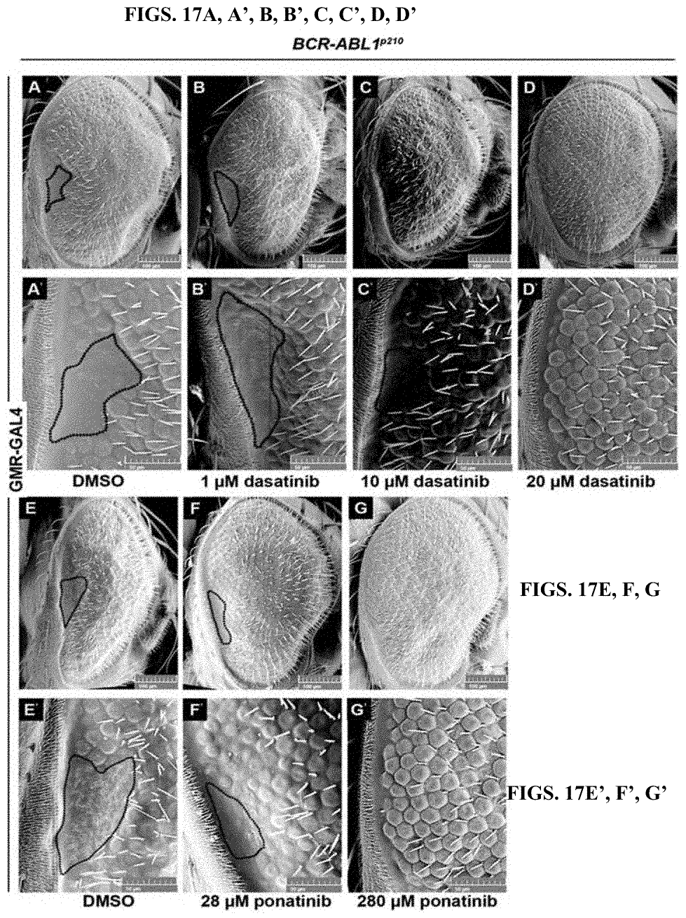

A dose-response analysis for BCR-ABL1^{p210} expressing flies treated with dasatinib showed a significant decrease in the average posterior eye defect area from about 4580 μm² in untreated flies to about 2372 μm² (P<0.0001) with 1 μM dasatinib, to about 131 μm² (P<0.0001) with 10 μM and to about 0 μm² (P<0.0001) with 20 μM dasatinib. The percentage of flies with total rescue increased from 25% to 92% and to 100% with 1 μM, 10 μm and 20 μM dasatinib respectively (FIG. 17). Similarly, ponatinib also showed a dose-response whereby the average posterior eye defect area decreased significantly from about 4044 μm² in untreated flies to about 1684 μm² (P<0.0001) with 28 μM and to about 267 μm² (P<0.0001) with 280 μM ponatinib (FIG. 17). The percentage of flies with total rescue increased from 48% to 86% with 28 μM and 280 μM ponatinib respectively.

BCR-ABL1^{p210/T315I} mutation is known to exhibit resistance to imatinib, nilotinib and dasatinib in CML patients and this was confirmed in the model system whereby the characteristic posterior eye defect did not show ommatidial rescue when feeding BCRABL1^{p210/T315I} expressing flies imatinib (FIG. 14 K-K′-L-L′), dasatinib (FIG. 15 H-H′) or nilotinib (FIG. 18 K-K′-L-L′). However, feeding ponatinib to BCR-ABL1^{p210/T315I} expressing flies did not show the expected rescue of the posterior eye defect (FIG. 16H-H′). Western blot analysis confirmed the expression and phosphorylation of BCR-ABL1^{p210} and BCR-ABL1^{p210/T315I} in Drosophila eyes from untreated or treated flies (FIGS. 15J-16J).

Tyrosine Kinase Inhibitors Screening Using Lethality Phenotype

Upon expression of BCR-ABL1^{p210} in Drosophila imaginal discs using engrailed-GAL4, F1 progeny showed mainly pupal lethality while BCR-ABL1^{p210/T315I} revealed earlier lethality of embryonic/larval stages at 25° C. indicating a more severe phenotype. Dasatinib (20 μM) and ponatinib (100 μM) was tested and showed reversal of rough eye phenotype could reverse pupal lethality and lead to eclosure of adult flies expressing BCR-ABL1^{p210}. Ponatinib (100 μM) was tested to show whether it could reverse larval lethality in BCR-ABL1^{p210/T315I} expressing flies.

Dasatinib and Ponatinib Reverse BCR-ABL1^{p210} Mediated Pupal Lethality

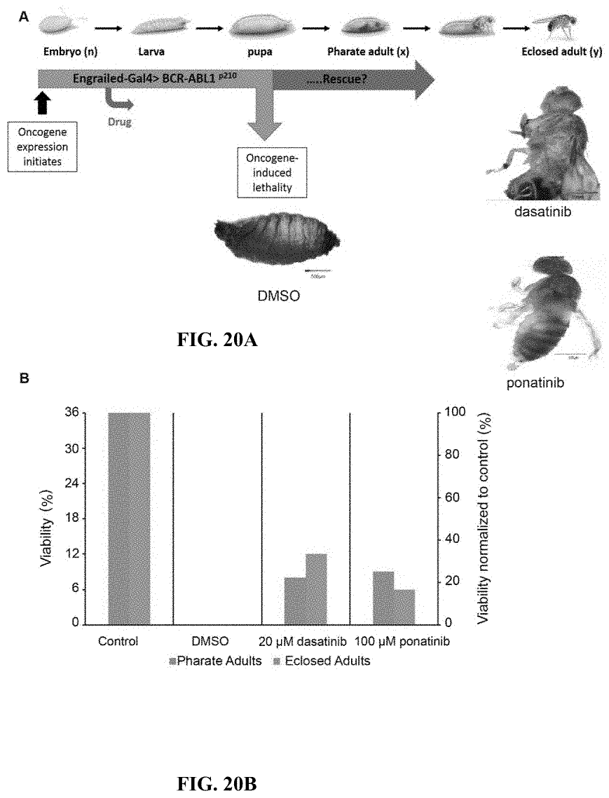

For quantification of the rescuing effects of dasatinib and ponatinib in BCR-ABL1^{p210} expressing flies under the control of engrailed-GAL4, embryos expressing BCR-ABL1^{p210} were transferred to food mixed with DMSO (0.1%), dasatinib (20 μM) or ponatinib (100 μM) then the percentage of eclosing adults or pharate adults was reported and normalized to percentage of surviving adult control flies (engrailed-Gal4>w¹¹¹⁸). Control flies showed a survival of 35% of eclosed adults while BCR-ABL1^{p210} expressing flies did not show any eclosing or even pharate adults on DMSO. On the other hand, treatment of BCR-ABL1^{p210} flies with 20 μM dasatinib resulted in a major developmental shift towards the adult stage whereby 32% of the flies eclosed normally out of their pupal cases as well as 21% formed pharate adults which are adults formed inside the pupal case but failed to eclose. Treatment with 100 μM ponatinib resulted in 17% eclosing adults and 25% paharate adults (FIGS. 20A-B).

Ponatinib Reverses BCR-ABL1^{p210/T315I} Mediated Larval Lethality

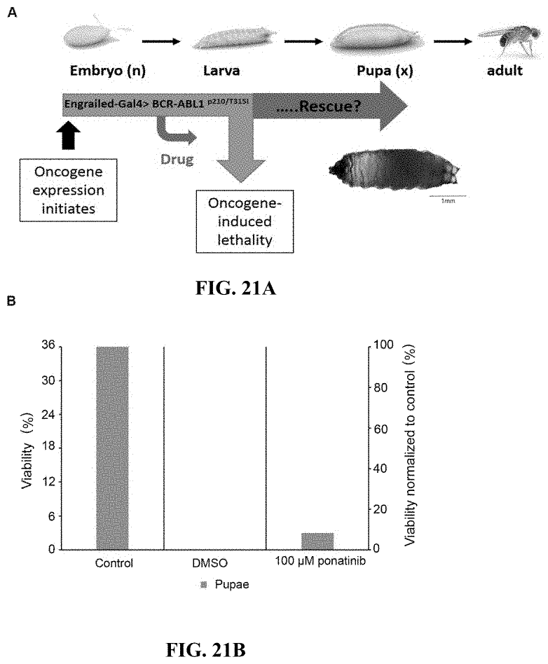

Embryos expressing BCR-ABL1^{p210/T315I} under the control of engrailed-GAL4 were transferred to food mixed with 0.1% DMSO or 100 μM ponatinib and the percentage of pupae forming was determined for each condition and normalized to percentage of pupae in control. No formation of pupae was detected in untreated flies however the developmental block at larval stage was partially reversed upon treatment with 100 μM ponatinib (FIGS. 21A-B).

In this embodiment, a transgenic Drosophila model expressing human BCRABL1 comprises a credible platform for CML drug screening. Contrary to what has been done previously by Fogerty et al. where chimeric human/fly BCR-ABL1 was expressed in Drosophila³³, this embodiment expressed a full human BCR-ABL1^{p210} protein. In a recent study, a CML Drosophila model expressing the human BCR-ABL1^{p210} was used to study genes and pathways that play a role in CML onset and progression³⁴.

The Drosophila eye, with its highly organized reiterative ommatidial structure, constitutes an efficient and relatively easy read out capable of amplifying subtle changes caused by disturbance to normal development. Therefore, this epithelial monolayer is a target tissue for expressing human BCR-ABL1^{p210} and human BCR-ABL1^{p210/T315I}. Bernardoni et al. 34 recently showed that expression of human BCRABL1^{p210} in Drosophila eyes was destructive to the normal eye development and resulted in a “glazy” eye phenotype as demonstrated by light microscopy images. The effect of increased temperature on transgene expression was investigated as well as used SEM analysis in addition to light microscopy to show the subtle details of the eye phenotypes. Moreover, one of the most elusive BCR-ABL1 mutations (T315I) behaves similarly or differently to the wild type variant. In this embodiment, with increased temperature, the rough eye phenotype was more prominent in T315I mutant BCR-ABL1, as shown in FIGS. 11-12. To validate the system for treatment screening, a specific area in the posterior end of the eye was evident to be defective in both BCR-ABL1^{p210} and BCR-ABL1^{p210/T315I} expressing flies. BCR-ABL1^{p210/T315I} expressing flies showed more severe phenotype characterized by a wider defective area of lost ommatidial facets as compared to flies expressing the wild type variant BCR-ABL1^{p210} indicating that the transformation capacity of T315I is much higher than the wild type BCR-ABL1^{p210}. Similar results were obtained when expressing BCR-ABL1^{p210/T315I} in other tissues where more detrimental effects were seen when compared to BCR-ABL1^{p210}. For example, expression of BCR-ABL1 in the fly imaginal discs resulted in pupal lethality with BCR-ABL1^{p210} expressing flies versus embryonic/larval lethality with BCR-ABL1^{p210/T315I} expressing flies (FIGS. 20-21).

The model system further comprises assessing the capability of the conventional treatments used in clinics for CML patients in improving the eye defects observed in the adult eyes of BCR-ABL1^{p210} and BCR-ABL1^{p210/T315I} flies. These TKIs include imatinib as first generation TKI, nilotinib and dasatinib as second and ponatinib as third generation TKI. Dasatinib and ponatinib resulted in the full rescue of BCR-ABL1^{p210} eye defect (FIGS. 15-16) in 100% and 86% of flies respectively. Imatinib and nilotinib (FIG. 14; FIG. 18) exhibited a lower percentage of rescue, 21% and 13% respectively; this might be attributed to the difference in drug potencies among imatinib and other TKIs whereby compared to imatinib, dasatinib exhibits 325-fold higher potency for BCR-AB1L inhibition in vitro whereas nilotinib is only 20 folds more potent²³. The limited rescuing efficacy of imatinib and nilotinib could be due to activation of dAbl by BCR-ABL1 expression shown previously by Bernardoni et al.,³⁴ where it was demonstrated that human BCR-ABL1 expression interferes with dAbl signaling pathway and increases Ena phosphorylation, a dAbl target. On the other hand, using Drosophila wing epithelium as an in vivo model, Singh et al.⁴¹ demonstrated that activated dAbl exerts a positive feedback loop on Drosophila Src members leading to an increase in their activity and hence signal amplification. Both dAbl 42 and Drosophila Src 43 play important roles in Drosophila eye development; therefore it is possible that upon human BCR-ABL1 expression in Drosophila eyes, the dAbl signaling pathway is activated, which in its turn activates Drosophila Src members and amplifies BCR-ABL1 mediated effects. In one embodiment, Src is one of the kinases inhibited by dasatinib and ponatinib but not imatinib and nilotinib, therefore, this explains the more robust rescuing effect seen by dasatinib and ponatinib. Dasatinib demonstrated target specificity in vivo whereby BCR-ABL1^{p210/T315I} flies fed on dasatinib showed the expected resistance to treatment. BCR-ABL1^{p210/T315I} resistance to imatinib and nilotinib was also confirmed as there was no rescue of ommatidial development. In contrary to what was expected, ponatinib was not successful in rescuing progeny expressing BCR-ABL1^{p210/T315I} The eye defect area was significantly larger upon BCRABL1^{p210/T315I} expression as compared to that of BCR-ABL1^{p210}. Noting this significant increase in the average posterior eye defect area, the phenotype was still very severe to allow for any drug reversal. Moreover, noting that the choice of the dose was limited by DMSO toxicity, ponatinib dose used may not have been high enough to reverse the defect. On the other hand, ponatinib was tested in rescuing the lethality phenotype of BCR-ABL1^{p210/T315I} flies; feeding ponatinib to BCR-ABL1^{p210/T315I} expressing flies rescued larval lethality and allowed development to the pupal stage which suggests that the drug's response is tissue dependent. As well as, feeding ponatinib or dasatinib to BCR-ABL1^{p210} expressing flies resulted in the rescue of pupal lethality and eclosure of adult flies (FIGS. 20-21). The in vivo model for BCR-ABL1 driven transformation is established, where the efficacy of the current potent treatments is shown by reversing a very subtle phenotype in a specific location in the posterior end of the adult compound eye. In another embodiment, this system assesses the efficacy of novel compounds by performing high throughput library testing in vivo. A Drosophila CML model system is enabled to screen for potential compounds especially that TKIs which are currently used do not target CML stem cells and hence are not curative.

Future Applications:

1. A library of compounds and natural products will be tested for identifying the effectiveness of potential targets in reversing the obtained detrimental phenotypes (mainly lethality phenotype). An example of treatment strategy that could be performed is testing of TKIs in combination with other drugs that can target CSCs such as Hedgehog pathway inhibitors. Moreover, the described models can be used to screen for their sensitivity to epigenetic reprogramming by drugs. One embodiment includes the Anti-cancer Compound Library from Selleckchem that contains 1128 anti-cancer small molecules which are inhibitors of PI3K, HDAC, mTOR, CDK, Aurora Kinase, JAK, etc. Contents of the library can be found from the following web site, http://www.selleckchem.com/screening/anti-cancer-compound-library.html.

2. A personalized approach includes specific mutations (single and compound) arising in CML patients will be screened for available and newly discovered single and combination treatment modalities. Multiple mutations can be created using the CRISPR/Cas9 system in Drosophila.

3. Identify/discover whether T315I mutation, as well as other de novo mutations, confer any additional severity in the transformation potential of BCR-ABL whereby a transcriptome analysis can be done to detect a difference in gene expression between wild-type BCR-ABL and mutated T315I or any other mutations in CML.

4. Perform a modifier genetic screen on the above-mentioned models to identify target pathways, genes and regulatory mechanisms that might be inhibited/activated by transformation and/or administration of selected compounds and inhibitors.

The CML Drosophila model described herein is responsive to the two potent TKIs dasatinib and ponatinib and is a needed piece of the puzzle in CML field whereby in contrast to CML mouse models, where it is a daunting process to screen for a large number of drug compounds, the CML model accomplishes this easily. In comparison to in vitro and in silico tools in CML drug discovery, the CML model described herein preserves the in vivo environment and gives a better idea of the drug response. The advantages of Drosophila in drug discovery are well known but the CML Drosophila model described herein is validated, which can fill the gaps in the traditional drug discovery approaches in CML. The special feature of the disclosed system is a lethality phenotype with a human BCR-ABL expression which can be reversed with TKIs and hence can be tested for reversal with other drugs or genetic epistasis experiments.

The main problem in CML disease is the need for curative CML stem cell-targeting drugs. Until such drugs can be within the hands of patients, TKIs remains the gold standard but with the expense of taking them for a lifetime. Moreover, resistance to treatment continually outshines the success of any developed treatment especially with TKIs which are taken for a long period of time without being eradicative of CSCs and hence allowing for the accumulation of mutations. Currently, patients facing resistance have the options of increasing the dosage of drugs with more side effects or shifting to newer generation drugs or reserving to ponatinib in case of T315I mutation. However ponatinib, in particular, is accompanied by serious side effects.

The Drosophila CML model disclosed herein can be exploited for:
Compound Library screening for targets that can efficiently reverse pupal lethality in BCR-ABL (wild-typep 210) and larval lethality in BCR-ABL (p210^{T315I}). Those targets can be later validated in other in vitro and in vivo CML mice models. One of the CML mice models that can be used for validating the drug hits in the Drosophila CML model system is the murine bone marrow retroviral transduction/transplantation model (4, 5). This approach might unravel targets that can prove in other models to be CML stem cell-targeting drugs and hence curative.
Screening for treatments for other single and compound mutations in CML.
Genetic modifier screening for genes that aggravate or reverse the lethality phenotype and hence provide a list of genes that play a role in pathogenesis and hence potential therapeutic targets. This can also be done in combination with feeding TKIs or other drugs to flies to test the response to the drug in the presence of the modifier screen.
BCR-ABL (wild-type p210) showed mainly pupal lethality with engrailed-driven expression whereas BCR-ABL (p210^{T315I}) showed lethality at earlier stages (embryonic/larval) therefore this model to can also be used to identify whether T315I mutation confers any additional severity in the pathogenesis. This can be extended to other mutations as well.

Thus, the embodiments disclosed herein substantially contribute to the field of CML in general and drug discovery in particular by providing the first CML Drosophila lethality phenotype model that can be efficiently used for drug and genetic screening.

Screening

High-throughput screening (HTS) of collections of chemically-synthesized molecules and of natural products (such as microbial fermentation broths) has traditionally played a central role in the search for lead compounds for the development of new pharmacological agents. Moreover, the potent and specific biological activities of many low molecular weight peptides make these molecules attractive starting points for therapeutic drug discovery (see e.g., Hirschmann, et al., 1991. Angew. Chem. Int. Ed. Engl. 30:1278-1301). The present invention discloses a methodology for the screening of compounds for desirable biological/therapeutic activities which involves the screening of individual chemical compounds which have been synthesized and cataloged in libraries of drug or chemical companies or research institutes. The active “lead” compounds and novel chemical entities identified and characterized by the present invention may be utilized for the development of bioactive “leads” in small molecule libraries for pharmaceutical, agrochemical and the like.

With respect to the generation of the small molecular weight compound libraries of the present invention, the combination of biochemical diversity is often synergistic with the metabolic diversity obtained from the in vivo production of “natural products”. Collections of naturally or synthetically produced chemical or oligomeric compounds, for example peptides, can be administered to cultures of microorganisms. In accord, each microbial strain may potentially create numerous modified chemical derivatives, thus generating a “metabolite library”. Because each of these aforementioned cultures would (potentially) contain a very complex mixture of metabolites, a highly efficacious method of screening would be required (i.e., HTS). An aliquot of the library is incubated with each of the many strains typical of a microorganism fermentation screening program, and the media screened utilizing an HTS-based assay. In another aspect of the invention, natural product diversity is screened by creating a mixture of combinatorially-tagged liposomes; wherein each liposome preferable encapsulates only one member or a simple mixture of a natural product compound library. The libraries which are generated by the methodologies disclosed herein may be screened for any biological activity known within the art. These include, but are not limited to: anti-microbial activity, anti-tumor activity, enzyme inhibiting activity, receptor binding, growth promotion activity, and in vitro and in vivo tests for biological responses.

With the advent of genomics, combinatorial paradigms and high-throughput screening (HTS) assay-based pharmacological testing, the number of compounds possessing biological/therapeutic activity is likely to markedly increase. HTS assays are, preferably, based upon automation, validation and integration of in vitro absorption-metabolism models and database management (see e.g., Rodrigues, et al., 1997. Phaim. Res. 14:1504-1510). Complementary to this tenet is the need to generate a taxonomy of known compounds, identifying those with similar mechanisms, preferably in a way that provides clues as to the nature of those mechanism.

The present invention discloses a screening methodology which is based upon the utilization of genetically-sensitized Drosophila systems with a new method for drug administration which permits both high-throughput and automation. Based upon previous experience with genetic screens for dominant modifier mutations, genetically-sensitized systems comprising specific pathways which are sensitive to such modifier mutations will permit detection of mere two-fold effects of small molecular weight compounds on specific signaling pathways related to human diseases. This assay sensitivity results from the ability to differentiate dose-dependent phenotypes of the modified Drosophila related to the presence of none, one, or two modified alleles within its genome. The methodology disclosed by the present invention is both more powerful and more sensitive than traditional, cell-free or in vitro cell-based assay systems. In addition, the Drosophila-based screening assay disclosed herein is based upon a biological “readout” related to human disease, and its intrinsic ability to test for both the overall specificity and toxicity of a compound(s) of interest.

It is contemplated that any signaling pathway may be utilized in the present invention. Many, if not all, signaling pathways are present in every living cell. However, only a subset of pathways are active at any given time in development. The present invention would deliver small molecule compounds to be tested at the time of development when the activity of the desired pathway is required. In Drosophila, the most sensitive phenotypic changes occur in the developing imaginal discs in the third instar larva, as most differentiation occurs at this stage.

As previously discussed, signaling pathways which play an important role in human diseases are the “targets” of this screening assay. Many, if not all, intracellular signaling pathways are indigenous to every cell. However, only a small subset are biologically active at any given time during cellular development and differentiation. Additionally, cell specificity is conferred upon activation of a specific signaling pathway by effector genes located downstream of a given pathway. It is these downstream effector genes which determines the cell- or tissue-specific phenotype, even though the signaling pathway may actually be indigenous to all cells. These signaling pathways may utilize intracellular “signal” which are normally turned on within the cells of interest, or they may utilize ectopic pathways. Ectopic expression of a component of a given pathway may be induced during development in either a temporally or spatially restricted manner. If the intact signaling pathway to be targeted is present within a cell which ectopically expresses the induced component, the component will activate the targeted signal transduction pathway. The prime target of a signaling pathway is ideally the component which is the most sensitive to alterations within the signaling pathway. The assay methodology of the present invention relies upon the screening of small molecular weight compounds to identify lead compounds that modify essential components in a given signaling pathway, resulting in an observably altered phenotype in comparison to untreated, wild-type Drosophila.

One experimental approach for the identification of new components of any given biological process is to search for mutations which dominantly modify the effects of another mutation disrupting the same biological process. This technique allows these aforementioned mutations to be recovered in a simple one-generation (F1) screening assay. More importantly, by sensitizing only a single biological pathway, one may create conditions in which even a slight reduction of gene activity (e.g., due to the loss of only one functional copy of the gene) can result in a detectable phenotype. This is of particular utility when the gene is involved in many other cellular processes, and a more severe loss of function may therefore produce an experimentally less-informative phenotype (e.g., such as lethality). This approach has been successfully used to identify components of the sevenless (sev) RTK signal transduction pathway involved in the induction of the R7 fate during eye development (see e.g., Simon, et al., 1991. Cell 67:701-716).

In one embodiment, the use of Drosophila strains which are genetically-modified within the Ras proto-oncogene signaling pathway. A component of this pathway, the Raf serine/threonine kinase, has been demonstrated to play a critical role in the signal transduction pathways activated by receptor tyrosine kinases (RTKs) across a broad phylogenetic spectrum. Within these signaling pathways, Raf acts to couple Ras activation to the mitogen-activated protein kinase (MAPK) cascade, which consists of the protein kinases MEK (MAPK kinase) and MAPK (see e.g., Marshall, 1995. Cell 80:179-185). The roles of these proteins in Raf signaling have been well-established by both biochemical and genetic studies. Less well-understood, however, are the roles of other Raf-binding proteins such as 14-3-3 proteins (see e.g., Fanti, et al., 1992. Nature 371:612-614; Freed, et al., 1994. Science 265: 1713-1716; Fu, et al., 1994. Science 266:126-129), hsp90 (see e.g., Stancato, et al., 1993. J. Biol. Chem. 268:21711-21716) and immunophilins (see e.g., Stancato, et al., 1994. J. Biol. Chem. 269:22157-22161), as well as the overall extent to which signal transduction via Raf is further regulated by as yet uncharacterized proteins acting within this, or parallel signaling pathways.

In genetically modified Drosophila strains, specific signaling pathways involved in human diseases can be activated at a threshold to produce an easily detectable altered phenotype. It is preferred that such strains are genotypic (+/null), and hence are hemizygous for a dose sensitive gene within a given signaling pathway. These gene products are prime candidates as targets for small compounds that interfere with their function.

Activation of the Ras/MAP kinase cascade by Torso RTK results in the specification of head and tail regions in embryonic cells while activation of the same cascade by the sev RTK in the developing eye results in the specification of photoreceptor cell fate. It is possible that each of these receptors activates specific pathways in addition to the common Ras/MAP kinase pathway. The cell-type specific response may depend on which of these parallel pathway is activated by a given receptor. In vertebrate cell culture systems, it has for example been shown that the platelet derived growth factor (PDGF) receptor activates multiple signaling pathways. Identification of the general signaling components downstream of sev, namely Torso and DER (Drosophila homolog of the EGF (epidermal growth factor receptor), is based on the study of loss-of-function (LOF) mutations, and indicate that the corresponding gene products thus identified are necessary for signaling. Indeed the complete removal of Drk, Sos, or Ras1 function in the Torso pathway produces a less severe phenotype than removal of either Torso or Raf function. This suggests that Torso can activate Raf independently of Drk, Sos and Ras1.

Additionally, Drosophila possessing the rolled gain of function mutation Sevenmaker (rlSEM) display a number of additional phenotypes that resemble those of gain of function mutations in torso and DER. Embryos derived from rlSEM females resemble those produced by females carrying a weak torsoGOF mutation since they lack to a variable degree the central segmented region. Similarly, the formation of extra veins on the wing of rlSEM flies is reminiscent of the Elp phenotype caused by a gain of function mutation in DER. Therefore, hyperactivation of MAP kinase is not only sufficient to activate the sev pathway but also the developmental pathways controlled by other RTKs.

The activation of RTK-specific signaling pathways that act in parallel to the general Ras/MAP kinase pathway is a possible way of maintaining the specificity of the inducing signal from the receptor to the nucleus. The decision of how a cell responds to the generic signal is taken in the nucleus and the nature of the response is determined to a large extent by the combination of nuclear factors present in the different cells at the time of induction. Hence, it may be an evolutionary advantage to use the same universal signaling cascade which can be activated by a number of different cell surface receptors to elicit a limited set of responses at any given stage in development and thereby successively restrict the developmental potential of cells.

It will be readily apparent to those individual skilled in the art that other genetically-modified Drosophila strains may be used in the practice of the present invention. An important attribute in choosing other appropriate Drosophila strains is that the strains have an easily monitored phenotype which is detectably-altered in response to the modification of genes related to disease pathways. Appropriate disease pathways include, but are not limited to, signaling pathways controlled by the Ras proto-oncogene; the WNT tumor suppressor gene; Rb (retinoblastoma tumor suppressor gene); HH (hedgehog development regulator) or the HH vertebrate homolog SHE (sonic hedgehog developmental regulator); activated protein kinase B (PKB/AKT); insulin receptor; insulin receptor substrates (IRS); c-src proto-oncogene; c-Jun proto-oncogene; c-myc proto-oncogene; p53; Janus kinases (JAK/STAT pathway); nitric oxide (NO); calmodulin; cAMP dependent protein kinase (PKA); Ca2+ dependent protein kinase (PKC); growth factors such as GH, TGF, PDGF and the like; receptor tyrosine kinases (RTKs); interferons (IFN); lipid metabolites; steroid hormones; phosphatidylinositol; G-protein coupled receptors; c-abl proto-oncogene; TGF-β and Smad gene family members; interleukins; GTPases; and ionophores. This list has been provided by way of example, for purposes of illustration only, and is not intended to be limiting with respect to scope, either real or implied.

The present invention discloses a methodology for the screening of genetically-modified Drosophila systems which are sensitive to gene dosage. The Drosophila phenotype which is to be assayed may involve the development of the eye, wing, or any other structure that develops from the imaginal discs in the fly. It should be noted that all previous assays to test the role of small molecular weight compounds have been performed with non-genetically-modified, wild-type Drosophila. Small molecular weight compounds will be administered by microinjection into the open circulatory system (i.e., hemolymph) of genetically-modified Drosophila third instar larvae for subsequent use in high-throughput screening (HTS) assays of small molecular weight compound libraries and/or in the identification of the dose response curve (or other pharmacological parameters) of lead compounds of interest.

The Drosophila third instar larval stage was chosen for both practical as well as specific considerations, which include, but are not limited to: (i) the third instar larvae are easily manipulated at this stage of development; (ii) the third instar larvae are large enough to facilitate automated microinjection in a high-throughput screening (HTS) assay; (iii) third instar larvae have an open circulatory system (i.e., hemolymph) through which there is rapid diffusion of the administered compound of interest; and (iv) in the third instar larvae myriad cellular signaling pathways are active in the growth and patterning of imaginal discs, which give rise to the adult structure(s). At this stage any one of these pathways can be genetically sensitized in a way that small perturbation in its activity lead to readily detectable phenotypes in the adult. Perturbation may occur by mutations in genes coding for essential components or as in the case of this invention, by selecting small compounds on the basis of their ability to specifically interfere the adult phenotype. Of equal importance is the fact that the myriad cellular signaling pathways are extremely active during this stage of Drosophila development due to imaginal disc development.

As previously discussed, the present invention discloses the utilization of a genetically-modified Drosophila strain which is specifically modified such that it possesses a gene (within a specific signaling pathway known to be involved in human diseases) which can be activated at a defined threshold level to produce an easily-detectable phenotype (e.g., the early pupal lethality, eye groove phenotype). This genetic modification may be produced by a naturally-occurring, non-wild-type allele of a specific gene which is isolated from a genetic mutagenesis screening assay well-known to those individual skilled within the art. Alternatively, the genetic modification may be produced by genetic manipulation using genetic recombination/molecular biological techniques known to those skilled within the art. The preferred genetic modification is a non-wild-type allele which, when present in the heterozygous state, results in an altered phenotype that is dose dependent. As utilized herein, the term “dose dependent” is designated as meaning that the genetically-sensitized Drosophila strain exhibits an observably different phenotype for each specific genetic state when it possesses either none, one or two copies of the modified allele of the gene of interest.

One embodiment utilizes the methodology of microinjection into the third instar larval developmental stage of genetically-modified Drosophila, thus permitting high-throughput screening (HTS) with subsequent automation of the assay procedure. This microinjection administration methodology offers several advantages over the conventional application by feeding. These advantages include, but not limited to: (i) both the time and developmental stage of the microinjection may be controlled in a precise manner; (ii) the amount, and thus the final concentration of the compound in vivo can be determined; (iii) the compound is rapidly dispersed through the open circulatory system and quickly reaches the target tissue, the imaginal discs; and (iv) the compounds are only administered at the time when the activity of the pathway is required to develop the easily scorable phenotype.

The HTS-based small molecular weight compound screening assay of the present invention is designed to ascertain (i.e., scores for) both the ability of the compound-treated larvae to undergo subsequent development (i.e., pupation) and for their eventual phenotype upon eclosion, in comparison to the mock-treated control Drosophila strain. The use of such in vivo assays allows for the highly relevant analysis of the bioavailability, biological/therapeutic function and toxicity of the compound(s) being tested. The present invention differs from conventional, HTS assays (based upon both cell-free or in vitro cell-based assay systems) in a number of ways including, but not limited to: (i) its overall degree of versatility; (ii) its indigenous screening capacity for compound specificity and non-toxicity and (iii) its lack of bias for specific classes of drug targets.

A wide variety of small molecular weight compounds may be used in the screen. Such compounds include, but are not limited to, any compositions which are being tested for lead drug discovery or development. Compounds may be aqueous- or lipid-soluble. Compounds may be delivered individually to separate Drosophila larvae or may be delivered to separate larvae as one of a plurality of different chemical compounds contained within a reagent solution, such as is performed within a multiplexing schema. Compounds may be dissolved or suspended within solution, or affixed to a solid-support. Solid supports may include, but are not limited to, insoluble polymer beads or a polymeric matrix coated with one or a plurality of individual compounds, or with combinatorial chemistries. Dosages and volumes which are microinjected into the Drosophila larvae may be varied so as to optimize dosages for further studies or to rank compounds as to their toxicity and/or potency. Information resulting from said variations in conditions may be used to prioritize chemical for further study, to delineate the relative toxicities of structurally related chemical, and/or to identify the proper dose range for subsequent toxicity studies (see e.g., Harris, et al., Fundam. Appl. Toxicol. 19:186-196).

In one embodiment of the present invention, recombinant DNA methodologies will be utilized to express exogenous genes which are functionally-linked to cell-specific transcriptional regulatory sequences. In an additional embodiment, exogenous genes which encode human homologs of the genes involved in the signaling pathway of interest will be utilized, so as to enable “humanization” of the aforementioned disease pathways. A preferred embodiment of the present invention involves the targeting of cell-specific expression of the incorporated exogenous genes to the cells of the Drosophila imaginal discs. Such genetic alterations possess the ability to greatly vary the genetic capabilities of the cells.

Compounds

Compounds which are screened by use of the methodology disclosed in the present invention may be useful as analgesics and/or for the treatment of inflammatory disease, especially in the case of the azotricyclic compounds acting as antagonists of the neurokin 1/brandykin receptor. Members of the benzodiazopine library may be useful as a muscle relaxant and/or tranquilizer and/or as a sedative. Members of the 23 million Mixed Amide Library may be of use in the treatment of hypertension on endothelin antagonists or Raynaud's syndrome.

The carbon-carbon backbone of the compounds of the present invention may be saturated or unsaturated, cyclic or linear. These aforementioned compounds include, but are not limited to, carbohydrates, polyalcohols (e.g., ethylene glycol and glycerol) and polyphenols (e.g., hydroquinones and tetracylines). Carbohydrate- and polysaccharide-transformed compounds are defined herein so as to include all chemical moieties possessing a saccharide unit or which are transformed from a saccharide. These compounds may also include glycopeptides, glycolipids and other biopolymers (or biomacromolecules) containing saccharides, either in their entirety or as part of the molecular framework. The term carbohydrates merely represent a portion of a much larger family of polyhydroxylated organic compounds which are within the scope of the present invention. In addition, the carbohydrated/polyhydroxylated organic compounds of the present invention include, but are not limited to: monomeric acyclic compounds (e.g., ethylene glycol, glycerol and 1,2,3-trihydroxy pentane); polymeric acyclic compounds (e.g., di- or tri-ethylene diglycol; monomeric cyclic compounds (e.g., inositol and 1,2,3-trihydroxycyclopentane); polymeric cyclic compounds (e.g., di-inositol); polymeric and monomeric unsaturated compounds (e.g., tetrahydroxy-1,4-quinone) and polyphenols (e.g., tetracyclines) and derivatives, analogs and fragments thereof.

With respect to the generation of the small molecular weight compound libraries of the present invention, the combination of biochemical diversity is often synergistic with the metabolic diversity obtained from the in vivo production of “natural products”. Collections of starting compounds, for example peptides, can be administered to cultures of microorganisms. In accord, each microbial strain may potentially create numerous modified peptides or peptide byproducts, thus generating a “metabolite library”. Because each of these aforementioned cultures would (potentially) contain a very complex mixture of metabolites, a highly efficacious method of screening would be required (i.e., HTS). An aliquot of the library is incubated with each of the many strains typical of a microorganism fermentation screening program, and the media screened utilizing an HTS-based assay. In another aspect of the invention, natural product diversity is screened by creating a mixture of combinatorially-tagged liposomes; wherein each liposome preferable encapsulates only one member or a simple mixture of a natural product compound library. The libraries which are generated by the methodologies disclosed herein may be screened for any biological activity known within the art. These include, but are not limited to: anti-microbial activity, anti-tumor activity, enzyme inhibiting activity, receptor binding, growth promotion activity, and in vitro and in vivo tests for biological responses. Compounds may be based on naturally occurring extracellular or intracellular signaling molecules or their derivatives or the like (see, e.g., Alberts, et al., 1989. “Chapter 12: Cell Signaling.” 2nd Edition. Garland Publishing, Inc., New York, N.Y., pp. 681-726).

Unfortunately, many peptidic-based compounds possess unfavorable pharmacodynamic properties such as poor oral bioavailability and rapid clearance in vivo, which have tended to limit the more widespread development of these compounds as potential therapeutic agents. This realization, however, has recently inspired workers to extend the concepts of combinatorial organic synthesis beyond peptide chemistry to create libraries of known pharmacophores (e.g., benzodiazepines; see e.g., Bunin, et al., 1992. J. Amer. Chem. Soc. 114:10997-10998), as well as polymeric molecules such as oligomeric N-substituted glycines (i.e., peptoids) and oligocarbamates (see e.g., Dower, et al., U.S. Pat. No. 5,639,603).

As used herein, the amino acid abbreviations are conventional one letter codes for the amino acids and are expressed as follows: A, alanine; B, asparagine or aspartic acid; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine.

“Peptide” as used herein refers to any peptide, oligopeptide, polypeptide, gene product, expression product, or protein. For example, a peptide can be an enzyme. A peptide is comprised of consecutive amino acids. The term “peptide” encompasses naturally occurring or synthetic molecules.

As used herein, the terms “transformation” and “transfection” mean the introduction of a nucleic acid, e.g., an expression vector, into a recipient cell including introduction of a nucleic acid to the chromosomal DNA of said cell. The art is familiar with various compositions, methods, techniques, etc. used to effect the introduction of a nucleic acid into a recipient cell. The art is familiar with such compositions, methods, techniques, etc. for both eukaryotic and prokaryotic cells. The art is familiar with such compositions, methods, techniques, etc. for the optimization of the introduction and expression of a nucleic acid into and within a recipient cell.

As used herein, the term “subject” refers to the target of administration, e.g., an animal. In an aspect, the subject of the herein disclosed methods can be a Drosophila. In an aspect, the subject of the herein disclosed methods can be mammal, a fish, a bird, a reptile, or an amphibian Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, the subject is a mammal. A patient refers to a subject afflicted with a disease or disorder. The term “patient” includes human and veterinary subjects. In some aspects of the disclosed methods, the subject has been diagnosed with a need for treatment for CML, such as, for example, prior to the administering step.

As used herein, the term “treatment” refers to the medical management of a subject or a patient with the intent to cure, ameliorate, stabilize, or prevent a disease, pathological condition, or disorder, such as, for example, CML. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as CML). In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the disease, pathological condition, or disorder is CML.

As used herein, the term “prevent” or “preventing” refers to precluding, averting, obviating, forestalling, stopping, or hindering something from happening, especially by advance action. In an aspect, prevent or preventing refers to the ameliorating of one or more signs and symptoms associated with CML. It is understood that where reduce, inhibit or prevent are used herein, unless specifically indicated otherwise, the use of the other two words is Also expressly disclosed.

As used herein, the term “diagnosed” means having been subjected to a physical examination by a person of skill, for example, a physician, and found to have a condition that can be diagnosed or treated by compositions or methods disclosed herein. For example, “diagnosed with CML” means having been subjected to a physical examination by a person of skill, for example, a physician, and found to have a condition that can be diagnosed or treated by a compound or composition that alleviates or ameliorates one or more symptoms associated with CML.

As used herein, the phrase “identified to be in need of treatment for a disorder,” or the like, refers to selection of a subject based upon need for treatment of the disorder. For example, a subject can be identified as having a need for treatment of a neurodegenerative disorder (e.g., CML) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. In an aspect, administering can refer to oral administration, such as, in food. Administration can be continuous or intermittent. In various aspects, a preparation can be administered therapeutically; that is, administered to treat an existing disease or condition. In further various aspects, a preparation can be administered prophylactically; that is, administered for prevention of a disease or condition, such as, for example, CML.

The term “contacting” as used herein refers to bringing a disclosed compound and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the term “determining” can refer to measuring or ascertaining a quantity or an amount or a change in expression and/or activity level, e.g., of a nucleotide or transcript or polypeptide. For example, determining the amount of a disclosed transcript or polypeptide in a sample as used herein can refer to the steps that the skilled person would take to measure or ascertain some quantifiable value of the transcript or polypeptide (i.e., BCR-ABL) in the sample. The art is familiar with the ways to measure an amount of the disclosed nucleic acids, transcripts, polypeptides, etc.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compares to a control (i.e., a non-affected or healthy subject or a sample from a non-affected or healthy subject) or a sham or an untreated sample).

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

By “modulate” is meant to alter, by increase or decrease. As used herein, a “modulator” can mean a composition that can either increase or decrease the expression level or activity level of a gene or gene product such as a peptide. In an aspect, a peptide can be BCR-ABL. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition.

In general, the biological activity or biological action of a peptide refers to any function exhibited or performed by the peptide that is ascribed to the naturally occurring form of the peptide as measured or observed in vivo (i.e., in the natural physiological environment of the protein) or in vitro (i.e., under laboratory conditions).

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Disclosed are the components to be used to prepare a composition of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

The invention does not preclude the use of any type of compound library. Each library has its own specific advantages and disadvantages.

### REFERENCES

1. Rowley J D. Letter: A new consistent chromosomal abnormality in chronic myelogenous leukaemia identified by quinacrine fluorescence and Giemsa staining Nature. 1973; 243 (5405):290-293.
2. Jain P, Kantarjian H, Patel K P, et al. Impact of BCR-ABL transcript type on outcome in patients with chronic-phase CML treated with tyrosine kinase inhibitors. Blood. 2016; 127(10):1269-1275.
3. Shepherd P, Suffolk R, Halsey J, Allan N. Analysis of molecular breakpoint and m-RNA transcripts in a prospective randomized trial of interferon in chronic myeloid leukaemia: no correlation with clinical features, cytogenetic response, duration of chronic phase, or survival. Br J Haematol. 1995; 89(3):546-554.
4. Groffen J, Stephenson J R, Heisterkamp N, de Klein A, Bartram C R, Grosveld G. Philadelphia chromosomal breakpoints are clustered within a limited region, bcr, on chromosome 22. Cell. 1984; 36(1):93-99.
5. Mandanas R A, Leibowitz D S, Gharehbaghi K, et al. Role of p21 RAS in p210 bcr-abl transformation of murine myeloid cells. Blood. 1993; 82(6):1838-1847.
6. Okuda K, Matulonis U, Salgia R, Kanakura Y, Druker B, Griffin J D. Factor independence of human myeloid leukemia cell lines is associated with increased phosphorylation of the proto-oncogene Raf-1. Exp Hematol. 1994; 22(11):1111-1117.
7. Raitano A B, Halpern J R, Hambuch T M, Sawyers C L. The Bcr-Abl leukemia oncogene activates Jun kinase and requires Jun for transformation. Proc Natl Acad Sci USA. 1995; 92(25):11746-11750.
8. Sawyers C L, Callahan W, Witte O N. Dominant negative MYC blocks transformation by ABL oncogenes. Cell. 1992; 70(6):901-910.
9. Shuai K, Halpern J, ten Hoeve J, Rao X, Sawyers C L. Constitutive activation of STAT5 by the BCR-ABL oncogene in chronic myelogenous leukemia. Oncogene.
1996; 13(2):247-254.
10. Carlesso N, Frank D A, Griffin J D. Tyrosyl phosphorylation and DNA binding activity of signal transducers and activators of transcription (STAT) proteins in hematopoietic cell lines transformed by Bcr/Abl. J Exp Med. 1996; 183(3):811-820.
11. Ilaria R L, Jr., Van Etten R A. P210 and P190(BCR/ABL) induce the tyrosine phosphorylation and DNA binding activity of multiple specific STAT family members. J Biol Chem. 1996; 271(49):31704-31710.
12. Druker B J, Talpaz M, Resta D J, et al. Efficacy and safety of a specific inhibitor of the BCR-ABL tyrosine kinase in chronic myeloid leukemia. N Engl J Med. 2001; 344(14):1031-1037.
13. O'Brien S G, Guilhot F, Larson R A, et al. Imatinib compared with interferon and low-dose cytarabine for newly diagnosed chronic-phase chronic myeloid leukemia. N Engl J Med. 2003; 348(11):994-1004.
14. Jabbour E, Kantarjian H M, Saglio G, et al. Early response with dasatinib or imatinib in chronic myeloid leukemia: 3-year follow-up from a randomized phase 3 trial (DASISION). Blood. 2014; 123(4):494-500.
15. Larson R, Hochhaus A, Hughes T, et al. Nilotinib vs imatinib in patients with newly diagnosed Philadelphia chromosome-positive chronic myeloid leukemia in chronic phase: ENESTnd 3-year follow-up. Leukemia. 2012; 26(10):2197.
16. Kantarjian H, Shah N P, Hochhaus A, et al. Dasatinib versus imatinib in newly diagnosed chronic-phase chronic myeloid leukemia. N Engl J Med. 2010; 362(24):2260-2270.
17. Kantarjian H M, Shah N P, Cortes J E, et al. Dasatinib or imatinib in newly diagnosed chronic-phase chronic myeloid leukemia: 2-year follow-up from a randomized phase 3 trial (DASISION). Blood. 2012; 119(5):1123-1129.
18. Jabbour E, Kantarjian HIM, Saglio G, et al. Early response with dasatinib or imatinib in chronic myeloid leukemia: 3-year follow-up from a randomized phase 3 trial (DASISION). Blood. 2014; 123(4):494-500.
19. Saglio G, Kim D W, Issaragrisil S, et al. Nilotinib versus imatinib for newly diagnosed chronic myeloid leukemia. N Engl J Med. 2010; 362(24):2251-2259.
20. Kantarjian H M, Hochhaus A, Saglio G, et al. Nilotinib versus imatinib for the treatment of patients with newly diagnosed chronic phase, Philadelphia chromosome-positive, chronic myeloid leukaemia: 24-month minimum follow-up of the phase 3 randomised ENESTnd trial. Lancet Oncol. 2011; 12(9):841-851.
21. Larson R A, Hochhaus A, Hughes T P, et al. Nilotinib vs imatinib in patients with newly diagnosed Philadelphia chromosome-positive chronic myeloid leukemia in chronic phase: ENESTnd 3-year follow-up. Leukemia. 2012; 26(10):2197-2203.
22. Lombardo L J, Lee F Y, Chen P, et al. Discovery of N-(2-chloro-6-methyl-phenyl)-2-(6-(4-(2-hydroxyethyl)-piperazin-1-yl)-2-methylpyrimidin-4-ylamino)thiazole-5-carboxamide (BMS-354825), a dual Src/Abl kinase inhibitor with potent antitumor activity in preclinical assays. J Med Chem. 2004; 47(27):6658-6661.
23. O'Hare T, Walters D K, Stoffregen E P, et al. In vitro activity of Bcr-Abl inhibitors AMN107 and BMS-354825 against clinically relevant imatinib-resistant Abl kinase domain mutants. Cancer Res. 2005; 65(11):4500-4505.
24. Tokarski J S, Newitt J A, Chang C Y, et al. The structure of Dasatinib (BMS-354825) bound to activated ABL kinase domain elucidates its inhibitory activity against imatinib-resistant ABL mutants. Cancer Res. 2006; 66(11):5790-5797.
25. Shah N P, Tran C, Lee F Y, Chen P, Norris D, Sawyers C L. Overriding imatinib resistance with a novel ABL kinase inhibitor. Science. 2004; 305(5682):399-401.
26. Weisberg E, Manley P W, Breitenstein W, et al. Characterization of AMN107, a selective inhibitor of native and mutant Bcr-Abl. Cancer Cell. 2005; 7(2):129-141.
27. Cortes J E, Kim D-W, Pinilla-Ibarz J, et al. Long-Term Follow-up of Ponatinib Efficacy and Safety in the Phase 2 PACE Trial. Blood. 2014; 124(21):3135-3135.
28. Cortes J E, Kim D W, Pinilla-Ibarz J, et al. A phase 2 trial of ponatinib in Philadelphia chromosome-positive leukemias. N Engl J Med. 2013; 369(19):1783-1796.
29. Breccia M, Pregno P, Spallarossa P, et al. Identification, prevention and management of cardiovascular risk in chronic myeloid leukaemia patients candidate to ponatinib: an expert opinion. Aim Hematol. 2017; 96(4):549-558.
30. Muller M C, Cervantes F, Hjorth-Hansen H, et al. Ponatinib in chronic myeloid leukemia (CML): Consensus on patient treatment and management from a European expert panel. Crit Rev Oncol Hematol. 2017; 120:52-59.
31. Mahon F-X, Réa D, Guilhot J, et al. Discontinuation of imatinib in patients with chronic myeloid leukaemia who have maintained complete molecular remission for at least 2 years: the prospective, multicentre Stop Imatinib (STIM) trial. Lancet Oncol. 2010; 11(11):1029-1035.
32. Gonzalez C. Drosophila melanogaster: a model and a tool to investigate malignancy and identify new therapeutics. Nat Rev Cancer. 2013; 13(3):172.
33. Fogerty F J, Juang J L, Petersen J, Clark M J, Hoffmann F M, Mosher D F. Dominant effects of the bcr-abl oncogene on Drosophila morphogenesis. Oncogene. 1999; 18(1):219-232.
34. Bernardoni R, Giordani G, Signorino E, et al. A new BCR-ABL1 Drosophila model as a powerful tool to elucidate pathogenesis and progression of chronic myeloid leukemia. Haematologica. 2019; 104(4):717-728.
35. Shirinian M, Kambris Z, Hamadeh L, et al. A Transgenic Drosophila melanogaster Model To Study Human T-Lymphotropic Virus Oncoprotein Tax-1-Driven Transformation In Vivo. J Virol. 2015; 89(15):8092-8095.
36. Schneider C A, Rasband W S, Eliceiri K W. NIH Image to Image: 25 years of image analysis. Nat Methods. 2012; 9(7):671-675.
37. Freeman M. Reiterative use of the EGF receptor triggers differentiation of all cell types in the Drosophila eye. Cell. 1996; 87(4):651-660.
38. Duffy J B. GAL4 system in Drosophila: a fly geneticist's Swiss army knife. Genesis. 2002; 34(1-2):1-15.
39. Xin S, Weng L, Xu J, Du W. The role of RBF in developmentally regulated cell proliferation in the eye disc and in Cyclin D/Cdk4 induced cellular growth. Development. 2002; 129(6): 1345-1356.
40. Cvetković V J, Mitrović T L, Jovanović B, et al. Toxicity of dimethyl sulfoxide against Drosophila melanogaster. Biol Nyssana. 2015; 6(2):91-95.
41. Singh J, Aaronson S A, Mlodzik M. Drosophila Abelson kinase mediates cell invasion and proliferation through two distinct MAPK pathways. Oncogene. 2010; 29(28):4033-4045.
42. Xiong W, Rebay I. Abelson tyrosine kinase is required for Drosophila photoreceptor morphogenesis and retinal epithelial patterning. Dev Dyn. 2011; 240(7):1745-1755.
43. Takahashi F, Endo S, Kojima T, Saigo K. Regulation of cell-cell contacts in developing Drosophila eyes by Dsrc41, a new, close relative of vertebrate c-src. Genes Dev. 1996; 10(13):1645-1656.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. An in vivo method of screening for a therapeutic for chronic myeloid leukemia, the method comprising: administering a candidate therapeutic for chronic myeloid leukemia to Drosophila larvae, wherein the larvae express a human BCR-ABL transgene; determining the survival of the larvae/pupae to a further developmental stage; and the survival of the larvae/pupae indicates that the candidate therapeutic for chronic myeloid leukemia is a therapeutic for chronic myeloid leukemia.

2. The method of claim 1, wherein the human BCR-ABL transgene comprises one or more mutations.

3. The method of claim 1, wherein the therapeutic for chronic myeloid leukemia ameliorates one or more signs or symptoms associated with chronic myeloid leukemia.

4. The method of claim 3, wherein the one or more signs or symptoms associated with chronic myeloid leukemia comprises phenotypic signs or symptoms.

5. The method of claim 1, further comprising examining eye and lethality phenotypes or a combination thereof.

6. A transgenic Drosophila comprising a human BCR-ABL gene.

7. The transgenic Drosophila of claim 6, wherein the BCR-ABL gene comprises one or more mutations.

8. The transgenic Drosophila of claim 6, and a therapeutic for CML identified by the transgenic Drosophila.

9. An in vivo method of screening for a therapeutic for CML, the method comprising: administering a candidate therapeutic for CML to Drosophila larvae, wherein the larvae express a human BCR-ABL and a p210^{T315I} transgene; and determining the rough eye phenotype (particularly the presence or absence of an eye groove); and the reversal of the rough eye phenotype (particularly the restoration of ommatidial development in the eye groove area) indicates that the candidate therapeutic for chronic myeloid leukemia is a therapeutic for chronic myeloid leukemia.

10. The method of claim 9, wherein the human BCR-ABL transgene comprises one or more mutations.

11. The method of claim 9, wherein the therapeutic for chronic myeloid leukemia ameliorates one or more signs or symptoms associated with chronic myeloid leukemia.

12. The method of claim 11 wherein the one or more signs or symptoms associated with chronic myeloid leukemia comprise phenotypic signs or symptoms including ommatidial fusions, misplaced mechanosensory bristles, and a characteristic groove at the lower end of the eye characterized by the loss of ommatidial facets.

13. A method of screening drug targets comprising:
Using a drosophila CML model as described in claim 1;
Screening a compound library for a plurality of targets that can efficiently reverse pupal lethality in BCR-ABL (wild-type p210) and larval lethality in BCR-ABL (p210^{T315I}); and
Validating the plurality of targets in at least one in vitro and in vivo CML mice models.

14. The method of claim 13, further comprising screening for treatments for other single and compound mutations in CML.

15. The method of claim 13, further comprising genetic modifier screening for genes that aggravate or reverse the lethality phenotype and to provide a list of genes that play a role in pathogenesis in CML.

16. The method of claim 15, further comprising feeding TKIs or other drugs to test the response to the drug in the presence of the modifier screen.

17. The method of claim 13, further comprising identifying whether T315I mutation confers any additional severity in the pathogenesis.

18. The method of claim 13, further comprising administering dasatinib to obtain a dose-response for BCR-ABL1^{p210} expressing flies treated with dasatinib showing a significant decrease in the average posterior eye defect area from about 4580 μm² in untreated flies to about 2372 μm².

19. The method of claim 13, further comprising administering ponatinib to obtain a dose-dependent response for BCR-ABL1^{p210} expressing flies treated with ponatinib showing a decrease in the average posterior eye defect area decreased significantly from 4044 μm2 in untreated flies to about 1684 μm² to about 267 μm².

20. The method of claim 13, further comprising administering imatinib, nilotinib and dasatinib to BCR-ABL1^{p210/T315I} mutation expressing flies, and the characteristic posterior eye defect does not show ommatidial rescue when feeding BCRABL1^{p210/T315I} expressing flies imatinib, dasatinib, or nilotinib; and administering ponatinib to BCR-ABL1^{p210/T315I} expressing flies does not show the rescue of the posterior eye defect.
